SEQUENCE LISTING (20 sequences)

SEQ ID NO: 1 - DNA, 426 bp, Artificial Sequence
  VH chain domain of clone 5B1
atggagtttgggctgagctggctttttcttgtggctattttaaaaggcgtacagtgccaggtgcagctggtggagtctgggggaggctcggtgcagcctggcaggtccctgagactctcctgtgaagcctctggattcacctttgaggcctatgccatgcactgggtccggcaacctccagggaagggcctggagtgggtctcaagtattaattggaatagtggtcgcatagcctatgcggactctgtgaagggccgattcaccatctccagagacaacgccaggaattccctgtatctgcaaatgaacagtctgagacttgaggacacggccttctattactgtgcaaaagatatacggaggtttagtaccgggggggcggagtttgagtactggggccagggaaccctggtcaccgtctcctca

SEQ ID NO: 2 - protein, 142 aa, Artificial Sequence
  VH chain domain of clone 5B1
MEFGLSWLFLVAILKGVQCQVQLVESGGGSVQPGRSLRLSCEASGFTFEAYAMHWVRQPPGKGLEWVSSINWNSGRIAYADSVKGRFTISRDNARNSLYLQMNSLRLEDTAFYYCAKDIRRFSTGGAEFEYWGQGTLVTVSS

SEQ ID NO: 3 - DNA, 390 bp, Artificial Sequence
  VL chain domain of clone 5B1
atggccggcttccctctcctcctcaccctcctcactcactgtgcagggtcttgggcccagtctgtgctgactcagccgccctcagcgtctgggacccccgggcagagggtcaccatctcttgttctggaagcagctccaacatcggaagtaattttgtatactggtaccagcagctcccaggaacggcccccaaactcctcatatataggaataatcagcggccctcaggggtccctgaccgattctctggctccaggtctggcacctcagcctccctggccatcagtggactccggtccgaggatgaggctgattattactgtgcagcatgggatgacagcctgggaggccattatgtcttcggaactgggaccaaggtcaccgtcctt

SEQ ID NO: 4 - protein, 130 aa, Artificial Sequence
  VL chain domain of clone 5B1
MAGFPLLLTLLTHCAGSWAQSVLTQPPSASGTPGQRVTISCSGSSSNIGSNFVYWYQQLPGTAPKLLIYRNNQRPSGVPDRFSGSRSGTSASLAISGLRSEDEADYYCAAWDDSLGGHYVFGTGTKVTVL

SEQ ID NO: 5 - DNA, 426 bp, Artificial Sequence
  VH chain domain of clone 9H3
atggagtttgggctgagctggctttttcttgtggctattttaaaaggcgtacagtgcgaagtgcagctgttggagtctgggggaggcttggtacagcctggcaggtccctgagactctcctgtgcggcctctggatttacctttgatgattatgtcatgcactgggtccggcaagctccagggaagggcctggagtgggtctcaagtattagttggaatagtggtagcataggctatgcggactctgtgaagggccgattcatcatctccagagacaacgccaagaactccctgtatctgcaaatgaacagtctgagagctgaggacacggccttgtattactgtgcaaaagatcgtcgtattaggggtgactcggggttcgagggtgactactggggccagggaaccctggtcaccgtctcctca

SEQ ID NO: 6 - protein, 142 aa, Artificial Sequence
  VH chain domain of clone 9H3
MEFGLSWLFLVAILKGVQCEVQLLESGGGLVQPGRSLRLSCAASGFTFDDYVMHWVRQAPGKGLEWVSSISWNSGSIGYADSVKGRFIISRDNAKNSLYLQMNSLRAEDTALYYCAKDRRIRGDSGFEGDYWGQGTLVTVSS

SEQ ID NO: 7 - DNA, 387 bp, Artificial Sequence
  VL chain domain of clone 9H3
atggccggcttccctctcctcctcaccctcctcactcactgtgcagggtcttgggcccagtctgtgttgacgcagccgccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgccagcctgagtggtgtggtattcggcggagggaccaagctgaccgtccta

SEQ ID NO: 8 - protein, 129 aa, Artificial Sequence
  VL chain domain of clone 9H3
MAGFPLLLTLLTHCAGSWAQSVLTQPPSASGTPGQRVTISCSGSSSNIGSNYVYWYQQLPGTAPKLLIYRNNQRPSGVPDRFSGSKSGTSASLAISGLRSEDEADYYCAAWDASLSGVVFGGGTKLTVL

SEQ ID NO: 9 - DNA, 426 bp, Artificial Sequence
  VH chain domain of clone 5H11
atggagtttgggctgagctggctttttcttgtggctattttaaaaggcgtacagtgccaggtgcagctgttggagtctgggggaggcttggtacagcctggcaggtccctgagactctcctgtgcagcctctggattcacctttgatgaatatgccatgcactgggtccggcaagctccagggaagggcctggagtgggtctcaagtgttagttggaatagtggtagcataggctatgcggactctgtgaagggccgattcaccatctccagagacaacgccaagaactccctgtatctacaaatgaacagtctgagagctgaggacacggccttgtattactgtgcaaaagatatacggacctatagcaccgggggggcggagtttgcctcctggggccagggaaccctggtcaccgcctcctca

SEQ ID NO: 10 - protein, 142 aa, Artificial Sequence
  VH chain domain of clone 5H11
MEFGLSWLFLVAILKGVQCQVQLLESGGGLVQPGRSLRLSCAASGFTFDEYAMHWVRQAPGKGLEWVSSVSWNSGSIGYADSVKGRFTISRDNAKNSLYLQMNSLRAEDTALYYCAKDIRTYSTGGAEFASWGQGTLVTASS

SEQ ID NO: 11 - DNA, 390 bp, Artificial Sequence
  VL chain domain of clone 5H11
atggccggcttccctctcctcctcaccctcctcactcactgtgcagggtcttgggcccagtctgtgttgacgcagccgccctcagcgtctgggacccccgggcagagggtcaccatctcttgttctggaagcagctccaacatcggaagtaattatgtatactggtaccagcaggtcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggccattatgtcttcggaactgggaccaaggtcaccgtccta

SEQ ID NO: 12 - protein, 130 aa, Artificial Sequence
  VL chain domain of clone 5H11
MAGFPLLLTLLTHCAGSWAQSVLTQPPSASGTPGQRVTISCSGSSSNIGSNYVYWYQQVPGTAPKLLIYRNNQRPSGVPDRFSGSKSGTSASLAISGLRSEDEADYYCAAWDDSLSGHYVFGTGTKVTVL

SEQ ID NO: 13 - DNA, 435 bp, Artificial Sequence
  VH chain domain of clone 7E3
atggagtttgggctgagctggctttttcttgtggctattttaaaaggcgtacagtgccaagtgcagctgttggagtctgggggaggcgtggtccagcctgggaggtccctgagactctcctgtgcagcctctggattcaccttcagtttctatggcatgcactgggtccgccaggctccaggcaaggggctggagtgggtggcagctatatcatatgatggaagtaataaatactatgcagactccgtgaagggccgattcaccatctccagagacaattccaagaacacgctgtatctgcaaatgaacagcctgagagctgaggacacggctgtgtattactgtgcgaaaaggcccaaccaattttattgtagtgatggtagatgctactccattgactactggggccagggaaccctggtcaccgtctcctca

SEQ ID NO: 14 - protein, 145 aa, Artificial Sequence
  VH chain domain of clone 7E3
MEFGLSWLFLVAILKGVQCQVQLLESGGGVVQPGRSLRLSCAASGFTFSFYGMHWVRQAPGKGLEWVAAISYDGSNKYYADSVKGRFTISRDNSKNTLYLQMNSLRAEDTAVYYCAKRPNQFYCSDGRCYSIDYWGQGTLVTVSS

SEQ ID NO: 15 - DNA, 390 bp, Artificial Sequence
  VL chain domain of clone 7E3
atggacatgagggtccccgctcagctcctggggctcctgctactctggctccgaggtgcccggtgtgaaattgtaatgacgcagtctccagccaccctgtctgtgtctccaggggagagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagacttcactctcaccatcagcagcctgcagtctgtagattctgcagtttattactgtcagcagtataataactggcctccgtacacttttggccaggggaccaagctggagatcaaa

SEQ ID NO: 16 - protein, 130 aa, Artificial Sequence
  VL chain domain of clone 7E3
MDMRVPAQLLGLLLLWLRGARCEIVMTQSPATLSVSPGERATLSCRASQSVSSNLAWYQQKPGQAPRLLIYGASTRATGIPARFSGSGSGTDFTLTISSLQSVDSAVYYCQQYNNWPPYTFGQGTKLEIK

SEQ ID NO: 17 - DNA, 750 bp, Artificial Sequence
  Diabody 5B1CysDb sequence
cagtctgtgctgacgcagccgccctcagcgtctgggacccccgggcagagggtcaccatctcttgttctggaagcagctccaacatcggaagtaattttgtatactggtaccagcagctcccaggaacggcccccaaactcctcatatataggaataatcagcggccctcaggggtccctgaccgattctctggctccaggtctggcacctcagcctccctggccatcagtggactccggtccgaggatgaggctgattattactgtgcagcatgggatgacagcctgggaggccattatgtcttcggaactgggaccaaggtcaccgtcctttctggtggtggtggtcaggtgcagctggtggagtctgggggaggctcggtgcagcctggcaggtccctgagactctcctgtgaagcctctggattcacctttgaggcctatgccatgcactgggtccggcaacctccagggaagggcctggagtgggtctcaagtattaattggaatagtggtcgcatagcctatgcggactctgtgaagggccgattcaccatctccagagacaacgccaggaattccctgtatctgcaaatgaacagtctgagacttgaggacacggccttctattactgtgcaaaagatatacggaggtttagtaccgggggggcggagtttgagtactggggccagggaaccctggtcaccgtctcctcaggttctcaccatcaccatcaccatggcggttgc

SEQ ID NO: 18 - protein, 250 aa, Artificial Sequence
  Diabody 5B1CysDb sequence
QSVLTQPPSASGTPGQRVTISCSGSSSNIGSNFVYWYQQLPGTAPKLLIYRNNQRPSGVPDRFSGSRSGTSASLAISGLRSEDEADYYCAAWDDSLGGHYVFGTGTKVTVLSGGGGQVQLVESGGGSVQPGRSLRLSCEASGFTFEAYAMHWVRQPPGKGLEWVSSINWNSGRIAYADSVKGRFTISRDNARNSLYLQMNSLRLEDTAFYYCAKDIRRFSTGGAEFEYWGQGTLVTVSSGSHHHHHHGGC

SEQ ID NO: 19 - DNA, 750 bp, Artificial Sequence
  Diabody 7E3CysDb sequence
gatgttgtgctgacgcagtctccagccaccctgtctgtgtctccaggggagagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagacttcactctcaccatcagcagcctgcagtctgaagattctgcagtttattactgtcagcagtataataactggcctccgtacacttttggccaggggaccaaggtggatatcaaatctggtggtggtggtgaagtgcagctggtggagtctgggggaggcgtggtccagcctgggaggtccctgagactctcctgtgcagcctctggattcaccttcagtttctatggcatgcactgggtccgccaggctccaggcaaggggctggagtgggtggcagctatatcatatgatggaagtaataaatactatgcagactccgtgaagggccgattcaccatctccagagacaattccaagaacacgctgtatctgcaaatgaacagcctgagagctgaggacacggctgtgtattactgtgcgaaaaggcccaaccaattttattgtagtgatggtagatgctactccattgactactggggccagggaaccctggtcaccgtctcctcaggttctcaccatcaccatcaccatggcggttgc

SEQ ID NO: 20 - protein, 249 aa, Artificial Sequence
  Diabody 7E3CysDb sequence
DVVLTQSPATLSVSPGERATLSCRASQSVSSNLAWYQQKPQAPRLLIYGASTRATGIPARFSGSGSGTDFTLTISSLQSEDSAVYYCQQYNNWPPYTFGQGTKVDIKSGGGGEVQLVESGGGVVQPGRSLRLSCAASGFTFSFYGMHWVRQAPGKGLEWVAAISYDGSNKYYADSVKGRFTISRDNSKNTLYLQMNSLRAEDTAVYYCAKRPNQFYCSDGRCYSIDYWGQGTLVTVSSGSHHHHHHGGC

(19) United States
(12) Patent Application Publication
(10) Pub. No.: US 2024/0190991 A1
(43) Pub. Date: Jun. 13, 2024

(54) COMBINATION THERAPY WITH AN ANTI-CA19-9 ANTIBODY AND FOLFIRINOX IN THE TREATMENT OF CANCER

(71) Applicant: BioNTech SE, Mainz (DE)

(72) Inventor: Hariz Hassan, Mainz (DE)

(21) Appl. No.: 18/283,715

(22) PCT Filed: Mar. 24, 2022
(86) PCT No.: PCT/EP22/57771

§ 371 Date: Sep. 22, 2023

Related U.S. Application Data
(60) Provisional application No. 63/166,885, filed on Mar. 26, 2021.

(51) Int. Cl. C07K 16/30; A61K 31/4745; A61K 31/513; A61K 31/519; A61K 31/555; A61K 39/00; A61K 45/06; A61K 47/68

(52) U.S. Cl. CPC C07K 16/303; A61K 31/4745; A61K 31/513; A61K 31/519; A61K 31/555; A61K 45/06; A61K 47/6859; A61K 2039/505; A61K 2039/545; C07K 2317/24; C07K 2317/31

(57) ABSTRACT

The present invention provides a combination therapy for effectively treating and/or preventing diseases associated with cells expressing CAI 9-9, including cancer diseases such as pancreatic cancer and metastases thereof.

The present invention relates to a combination therapy with an antibody having the ability to bind to CA19-9 and the chemotherapeutic agent FOLFIRINOX for the treatment of CA19-9-positive cancers and metastatic malignancies, such as pancreatic ductal adenocarcinoma, as well as to pharmaceutical compositions and kits comprising the antibody and agent.

### BACKGROUND

The Sialyl Lewis A (sLe^{a}) antigen is an epitope present on Carbohydrate Antigen 19-9 (CA19-9), that has been shown to be overexpressed on epithelial cell tumors (Magnani et al., 1982, J Biol Chem. 257:14365-14369; Magnani et al., 1983, Cancer Res. 43:5489-5492). sLe^{a} is an oligosaccharide expressed primarily as a proteoglycan that is secreted and circulates as a mucin form, and also as a less well studied glycolipid form (Magnani et al., 1983, Cancer Res. 43:5489-5492; Ringel et al., 2003, Mol Cancer 2:9). The sLe^{a} antigen is expressed predominantly on cancer cells (Kannagi et al., 2007, Chang Gung Med J. 30:189-209). As a ligand for E selectin, sLe^{a} facilitates tumor adhesion and extravasation, key events for tumor metastasis, and is thus a marker of an aggressive tumor phenotype (Sato et al., 1997, Anticancer Res. 17:3505-3511). Glycolipids, such as sLe^{a}, are established targets for cancer immunotherapies (Feizi, 1985, Nature 314:53-57). CA19-9 is widely expressed on tumors of the gastrointestinal tract, with up to 94% of pancreatic cancers positive for CA19-9 expression and high expression rates also seen in bile duct carcinomas and transitional cell carcinomas (Loy et al., 1993, Am J Clin Pathol. 99:726-728; Passerini et al., 2012, Am J Clin Pathol 138:281-287). Additionally, expression of CA19-9 is frequently seen in ovarian, colon, stomach, and distal esophagus/stomach cancers.

Circulating serum levels of CA19-9 have been validated as a biomarker for assessing the metastatic potential of pancreatic ductal adenocarcinomas (PDAC) (Ballehaninna and Chamberlain, 2012, J Gastrointest Oncol. 3(2):105-119; Dong, 2014, World J Surg Oncol. 12:171) and have been used to evaluate the aggressiveness of other epithelial cell cancers (Locker et al., 2006, J Clin Oncol. 24:5313-5327; Nakayama, 1995, Cancer 75:2051-2056). As a known ligand for endothelial leukocyte adhesion molecules, CA19-9 expression is associated with increased metastatic potential in colon cancer (Matsui et al., 2004, Jpn J Clin Oncol. 34:588-593; Ben-David, 2008, Immunol Lett. 116:218-224; Sato et al., 1997, Anticancer Res. 17:3505-3511) and pancreatic adenocarcinoma (Kishimoto et al., 1996, Int J Cancer 69:290-294). Serum CA19-9 levels have also been found to be informative with respect to prognosis and treatment effect in subjects with pancreatic cancer, with several studies correlating increasingly higher serum levels with poorer survival outcomes (Ballehaninna and Chamberlain, 2012, J Gastrointest Oncol. 3(2):105-119; Berger et al., 2004, Ann Surg Oncol. 11:644-649; Dong et al., 2014, World J Surg Oncol. 12:171). In a phase I/II clinical trial of nab-paclitaxel and gemcitabine in subjects with advanced pancreatic cancer, decreases in CA19-9 levels correlated with tumor response, PFS, and OS (Von Hoff et al., 2011, J Clin Oncol. 29:4548-4554). In a phase II study of 5-fluorouracil-based chemoradiotherapy in subjects with locally advanced pancreatic cancer, a greater than 90% reduction in CA19-9 levels from baseline was associated with significantly improved median survival time, with a multivariate analysis finding a-post therapy CA19-9 level of less than 85.5 u/mL to be an independent prognostic factor for survival (Yang et al., 2013, J Gastrointest Oncol. 4:361-369).

Serum CA19-9 levels may also be informative in other tumor types. In subjects with hepatocellular carcinoma, elevated CA19-9 levels were associated with increased mortality (Hsu et al., 2015, Clin Transl Gastroenterol. 6:e74). In a study of 43 breast cancer subjects with infiltrating ductal carcinoma, sLe^{a} was found in 79% of specimens, with higher levels of expression correlating with greater nodal involvement (Steplewska-Mazur et al., 2000, Hybridoma 19:129-133).

Pancreatic ductal adenocarcinoma (PDAC) is one of the most aggressive and difficult to treat human cancers. In 2015, there were an estimated 46,960 new cases of PDAC diagnosed in the United States and 40,560 deaths from this disease (NCI 2015). Despite best available therapies, the 5-year overall survival rate remains a dismal 7.2%, a rate that has remain essentially unchanged since 1975 (NCI 2015). Currently, pancreatic cancer accounts for 3% of all newly diagnosed cancers in the United States, a figure that continues to rise, and is responsible for 7% of all cancer deaths. For the small percentage of subjects diagnosed with early stage pancreatic cancer, a cure may be possible with surgery; however, approximately 90% of subjects initially present with advanced/unresectable disease (NCI 2015). Stage at diagnosis is prognostic for survival, though even subjects with localized disease and the best prognosis tend to have poor outcomes, with a 5-year survival rate of only 27%.

Pancreatic cancer is considered to be resistant to most available chemotherapy and irradiation regimens. Response to immunotherapies has been poor, possibly related to the presence of thick stroma surrounding the tumor, which has, until recently, rendered immunotherapy ineffective (Brower, 2014, J Natl Cancer Inst. 106(12)).

There have been modest improvements in treatment options for subjects with metastatic pancreas adenocarcinoma. The FOLFIRINOX chemotherapy regimen demonstrated improvements in tumor response, progression-free survival (PFS) and overall survival (OS) benefit compared to single agent gemcitabine (Conroy et al., 2011, N Engl J Med. 364:1817-1825). More recently, the combination of nab-paclitaxel (nanoparticle albumin-bound paclitaxel; Abraxane®) and gemcitabine demonstrated improvements in tumor response rate and PFS, with an OS benefit of approximately 2 months compared with gemcitabine alone. Based on these data, the combination of nab-paclitaxel and gemcitabine is a current standard of care as first-line therapy in pancreatic cancer subjects with good performance status (von Hoff et al., 2013, N Engl J Med. 369:1691-1703). Still, substantial improvements in treatment outcomes for pancreatic cancer subjects remain elusive. New therapies that extend survival in the absence of significant toxicity would substantially impact the outcome and quality of life for subjects with this disease.

As described herein, the combination of an anti-CA 19-9 antibody and the chemotherapy regiment FOLFIRINOX results in a more than additive, i.e., synergistic effect in the treatment of a CA 19-9-positive cancer, such as pancreatic cancer.

### SUMMARY

The present disclosure demonstrates surprising effectiveness of a particular combination regimen in treatment of diseases, disorders and conditions associated with CA19-9 expression. For example, the present disclosure demonstrates synergistic benefits when a subject receives a treatment regimen comprising a combination of CA19-9-targeted antibody therapy and FOLFIRINOX. Such diseases, disorders and conditions associated with cells expressing CA19-9 include cancer diseases such as pancreatic cancer, gastric cancer, esophageal cancer, lung cancer such as non-small cell lung cancer (NSCLC), ovarian cancer, colon cancer, hepatic cancer, head-neck cancer, cancer of the gallbladder, and metastases of the foregoing, such as peritoneal metastasis and lymph node metastasis. In an embodiment, the cells expressing CA19-9 express the Sialyl Lewis A (sLe^{a}) antigen epitope present on CA19-9. In an embodiment, the cancer diseases are pancreatic cancer and metastases thereof, such as advanced or metastatic pancreatic ductal carcinoma (PDAC). The combination therapy comprises antibody therapy, in which a molecule or agent comprising the antigen-binding component of an antibody having the ability to bind to CA19-9, is administered in combination with FOLFIRINOX therapy. In some embodiments, CA19-9-targeted antibody therapy is administered to a subject who is receiving or has received therapy with FOLFIRINOX. In some embodiments, FOLFIRINOX therapy is administered to a subject who is receiving or has received therapy with a CA19-9-targeted antibody therapy. In an embodiment, the combination therapy can be where the antibody therapy and FOLFIRINOX are individually administered to the patient within one, two or three weeks of each other. In an embodiment, the combination therapy can be where the antibody therapy and FOLFIRINOX are individually administered to the patient within one, two or three months of each other. In an embodiment, the combination therapy is not where the antibody therapy and FOLFIRINOX are individually administered to the patient greater than three months apart.

Combination therapy as described herein surprisingly results in a more effective treatment than is achieved with the antibody or FOLFIRINOX alone. In fact, provided combination treatment results in a more than additive effect, e.g., synergistic treatment effect. In an embodiment, the more than additive or synergistic effect can be observed/measured by one or more of the RECIST or iRECIST responses and the durability of such responses, e.g., those described in Seymour et al., 2017, Lancet Oncol. 18:e143-e152. For example, a synergistic decrease in size and/or volume of the tumor and/or decrease in the numbers of individual (metastatic) tumors is observed in a cancer patient who has undergone the combination therapy. For example, the more than additive effect can be seen by the longer overall survival, by the longer progression-free survival, and/or by the longer freedom from disease progression (stable disease state). For example, the more than additive effect also can be reflected in the reduction of tumor-related symptoms, such as cancer-related pain or a reduced need for pain medications during and following therapy. For example, the more than additive effect also can be reflected by an improved measure of patient quality of life, such as patient mobility, strength of appetite, psychological status. The more than additive or synergistic effect also can be observed by a significant lowering of the dose of the antibody and/or one or more components of FOLFIRINOX administered to the patient in the first or subsequent cycles of treatment in order to achieve the same treatment effect when given individually, e.g., the doses of either or both are lowered by at least 20%, 25%, 30%, 35%, 40%, 45%, 50%, 55%, 60% or more.

Without being bound by a particular mechanism, it is believed that the administration of FOLFIRINOX, i.e., providing a patient with the FOLFIRINOX therapy regimen, results in a significant reduction in the amount of circulating CA19-9 antigen, such that the antibody therapy, e.g., a molecule or agent having the ability to bind to CA19-9, for example those specifically described herein, are able to primarily bind to the CA19-9-positive tumor cells, such that a more cytotoxic effect on the tumor cells is obtained.

In one aspect, the present invention is directed to a method of treating or preventing a CA19-9-positive cancer in a patient comprising administering to the patient an antibody having the ability to bind to CA19-9 in combination with the administration of FOLFIRINOX. As used in the context of the present invention, the term “antibody having the ability to bind to CA19-9” encompasses any molecule or agent which has the ability to bind to CA 19-9, and in particular includes molecules or agents that comprise the antigen binding fragment of an antibody having the ability to bind CA 19-9. For example, encompassed within this term is a molecule that comprises the heavy and light chain variable regions of any of the monoclonal antibodies described herein.

In an aspect, the present invention is directed to a method of treating a disease, disorder or condition associated with CA19-9 expression with CA19-9-targeted antibody therapy, wherein the improvement comprises treating by administering the CA19-9-targeted antibody therapy in combination with FOLFIRINOX.

In an embodiment, the antibody having the ability to bind to CA19-9 can be administered repeatedly at a dose of up to 100 mg/kg, or can be administered repeatedly at a dose of 0.01 to 10 mg/kg. In an embodiment, the antibody having the ability to bind to CA19-9 can be administered repeatedly at a dose of 0.5 to 1.0 mg/kg.

In an embodiment, the antibody having the ability to bind to CA19-9 can be administered once a week, once every two weeks, once every three weeks, once every six weeks, or once every two months. In an embodiment, the antibody having the ability to bind to CA19-9 is administered once every two weeks.

FOLFIRINOX can comprise oxaliplatin, leucovorin, irinotecan and 5-fluorouracil, and in one embodiment FOLFIRINOX can be administered at a dose of 65 mg/m² oxaliplatin, 400 mg/m² leucovorin, 150 mg/m² irinotecan, and 1200 mg/m² 5-fluorouracil.

In an embodiment, the antibody having the ability to bind to CA19-9 is administered in an amount of 0.5 to 1.0 mg/kg once every two weeks starting on day 1 and FOLFIRINOX is administered intravenously at a dose of 65 mg/m² oxaliplatin on day 1, 400 mg/m² leucovorin on day 1, 150 mg/m² irinotecan on day 1, and a total of 1200 mg/m² 5-fluorouracil over days 1 and 2. After FOLFIRINOX treatment is complete, the antibody having the ability to bind to CA19-9 can be used as a monotherapy/maintenance therapy, which monotherapy/maintenance therapy can comprise administration once every week, once every two weeks, once every three weeks or once a month.

In an embodiment, the antibody having the ability to bind to CA19-9 can be administered after administration of all of the components of FOLFIRINOX, for example, after the 46-hour continuous infusion 5-fluorouracil administration.

In an embodiment, the antibody having the ability to bind to CA19-9 can be first administered following the first two weeks of FOLFIRINOX, i.e., on day 17 of the start of treatment.

In an embodiment, the antibody having the ability to bind to CA19-9 can be administered as a monotherapy after at least the first cycle of combination therapy.

In embodiment, the method further comprises administering to the patient concurrently or successively one or more additional agents. The additional agent can be a chemotherapeutic agent, e.g., selected from the group consisting of gemcitabine, paclitaxel, prodrugs thereof, salts thereof, and combinations thereof. The additional agent can be an immunotherapeutic agent, preferably an agent capable of stimulating γδ T cells, wherein the γδ T cells are preferably Vγ9Vδ2 T cells, for example, is a bisphosphonate or a nitrogen-containing bisphosphonate (aminobisphosphonate). The agent capable of stimulating γδ T cells can be selected from the group consisting of zoledronic acid, clodronic acid, ibandronic acid, pamidronic acid, risedronic acid, minodronic acid, olpadronic acid, alendronic acid, incadronic acid and salts thereof. In an embodiment, the agent capable of stimulating γδ T cells can be administered in combination with interleukin-2.

In an embodiment, the antibody having the ability to bind to CA19-9 can mediate cell killing by one or more of complement dependent cytotoxicity (CDC) mediated lysis, antibody dependent cellular cytotoxicity (ADCC) mediated lysis, induction of apoptosis and inhibition of proliferation.

In an embodiment, the antibody having the ability to bind to CA19-9 can be a human antibody.

In an embodiment, the antibody having the ability to bind to CA19-9 can be an antibody binding fragment selected from the group consisting of a Fab, a Fab′, a F(ab′)₂, a scFV, a diabody, a triabody, a minibody and a single-domain antibody (sdAB). In an embodiment, the antibody having the ability to bind to CA19-9 is a diabody, preferably comprising the amino acid sequence of SEQ ID NO: 18 or 20 (encoded by a polynucleotide having the nucleic acid sequence of SEQ ID NO: 17 or 19, respectively). In an embodiment, the antibody having the ability to bind to CA19-9 can be a polyclonal, monoclonal antibody or a chimeric antibody, optionally having an IgG or IgM isotype of any subclass, such as subclass I.

In an embodiment, the antibody having the ability to bind to CA19-9 can be an antibody conjugate, wherein the antibody conjugate comprises an antibody or fragment thereof having the ability to bind to CA19-9 covalently conjugated or recombinantly fused to another moiety, which moiety can be a stabilizing agent, a diagnostic agent, a detectable agent or a therapeutic agent.

In an embodiment, the antibody having the ability to bind to CA19-9 is MVT-5873 (also referred to herein as 5B1), a fully human IgG1 monoclonal antibody that targets the sialyl Lewis A (sLe^{a}), an epitope on CA19-9; see, e.g., Gupta et al., 2020, J Gastrointest Oncol 11:231-235). MVT-5873 comprises the VH and VL domains depicted in SEQ ID NO: 2 and 4, respectively.

In an embodiment, the antibody having the ability to bind to CA19-9 comprises a variable heavy chain (VH) domain having an amino acid sequence selected from the group consisting of residues 20-142 of SEQ ID NO: 2, residues 20-142 of SEQ ID NO: 6, residues 20-142 of SEQ ID NO: 10, and residues 20-145 of SEQ ID NO: 14. In an embodiment, the antibody having the ability to bind to CA19-9 comprises a variable light chain (VL) domain having an amino acid sequence selected from the group consisting of residues 20-130 of SEQ ID NO: 4, residues 20-129 of SEQ ID NO: 8, residues 20-130 of SEQ IP NO: 12, and residues 23-130 of SEQ ID NO: 16.

In an embodiment, the antibody having the ability to bind to CA19-9 comprises a variable heavy chain (VH) domain and a variable light chain (VL) domain, where said VH domain and said VL domain respectively comprise an amino acid sequence selected from the group consisting of residues 20-142 of SEQ ID NO: 2 and residues 20-130 of SEQ ID NO: 4; residues 20-142 of SEQ ID NO: 6 and residues 20-129 of SEQ ID NO: 8; residues 20-142 of SEQ ID NO: 10 and residues 20-130 of SEQ ID NO: 12; and residues 20-145 of SEQ ID NO: 14 and residues 23-130 of SEQ ID NO: 16.

In an embodiment, the antibody having the ability to bind to CA19-9 comprises a variable heavy chain (VH) domain comprising the amino acid sequence of residues 20-142 of SEQ ID NO: 2 and a variable light chain (VL) domain comprising the amino acid sequence of residues 20-130 of SEQ ID NO: 4.

In an embodiment, the antibody having the ability to bind to CA19-9 is an antibody selected from the group consisting of (i) an antibody which is a chimerized or humanized form of the antibody defined by the sequence identifiers above, (ii) an antibody having the specificity of the antibody defined by the sequence identifiers above, and (iii) an antibody comprising the antigen binding portion or antigen binding site, in particular the variable region, of the antibody defined by the sequence identifiers above and preferably having the specificity of the antibody defined by the sequence identifiers above.

In an embodiment, the antibody having the ability to bind to CA19-9 binds to the Sialyl Lewis A (sLe^{a}) antigen epitope present on CA19-9. In an embodiment, expression of CA19-9 can be on the cell surface of a cancer cell.

In an embodiment, the CA19-9-positive cancer is pancreatic cancer, for example, primary pancreatic cancer, advanced pancreatic cancer or metastatic pancreatic cancer, or a combination thereof such as a combination of pancreatic primary cancer and metastatic cancer.

In an embodiment, the pancreatic cancer comprises a cancer of the pancreatic duct, or the pancreatic cancer comprises an adenocarcinoma or carcinoma, or a combination thereof. In an embodiment, the pancreatic cancer comprises a ductal adenocarcinoma, a mucinous adenocarcinoma, a neuroendocrine carcinoma or an acinic cell carcinoma, or a combination thereof. In an embodiment, the pancreatic cancer is partially or completely refractory to gemcitabine treatment such as gemcitabine monotherapy.

In an embodiment, the pancreatic cancer is advanced or metastatic pancreatic ductal carcinoma (PDAC).

In an embodiment, the metastatic pancreatic cancer comprises metastasis to any one of the lymph nodes, ovary, liver, lung, or in any combination.

In an embodiment, the patient has a precancerous pancreatic lesion, in particular a precancerous pancreatic lesion comprising a beginning malignant histological change in the pancreatic ducts. In an embodiment, the patient has had surgery for the CA19-9-positive cancer.

In an embodiment, the patient has circulating levels of CA19-9 of less than 4000 U/mL, preferably less than 1000 U/ml, or the patient has circulating levels of CA19-9 of 37 U/ml or less, or no detectable circulating levels of CA19-9. The levels of CA19-9 can be measured any appropriate method known in the art, such as the electrochemiluminescence immunoassay (ECLIA) test by Labcorp Burlington, North Carolina. Such methods are also disclosed in Passerini et al., 2007, Clin Chem Lab Med 45:100-104 and in Ballehaninna and Chamberlain, 2011, Indian J Surg Oncol 2:88-100.

An aspect of the present invention is a combination medical preparation for treating or preventing a CA19-9-positive cancer comprising (i) an antibody having the ability to bind to CA19-9 and (ii) FOLFIRINOX. In an embodiment, the medical preparation can be present in the form of a kit comprising a first container including the antibody having the ability to bind to CA19-9 and a second container including FOLFIRINOX or one or more containers of one or more of the agents comprising FOLFIRINOX. In an embodiment, the medical preparation can further include printed instructions for use of the preparation for treatment or prevention of the CA19-9-positive cancer.

In an aspect, the present invention is directed to CA19-9-targeted antibody therapy for use in a method of treating a disease, disorder or condition associated with CA19-9 expression, wherein the method comprises administering the CA19-9-targeted antibody therapy in combination with FOLFIRINOX. In an aspect, the present invention is directed to FOLFIRINOX for use in a method of treating a disease, disorder or condition associated with CA19-9 expression, wherein the method comprises administering FOLFIRINOX in combination with CA19-9-targeted antibody therapy.

### DETAILED DESCRIPTION OF EMBODIMENTS OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

In the following, elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., (1995) Helvetica Chimica Acta, CH-4010 Basel, Switzerland.

The practice of the present invention will employ, unless otherwise indicated, conventional methods of biochemistry, cell biology, immunology, and recombinant DNA techniques which are explained in the literature in the field (cf, e.g., Molecular Cloning: A Laboratory Manual, 4th Edition, M. R. Green, J. Sambrook et al. eds., Cold Spring Harbor Laboratory Press, Cold Spring Harbor 2012).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e., the subject-matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The term “CA19-9” relates to Carbohydrate Antigen 19-9 and includes any modifications which can comprise the Sialyl Lewis A (sLe^{a}) antigen epitope.

With regard to nucleotide and amino acid sequences, the term “variant” according to the invention refers, in particular, to mutants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. A species homolog is a nucleic acid or amino acid sequence with a different species of origin from that of a given nucleic acid or amino acid sequence. The term “variant” shall encompass any post-translationally modified variants and conformation variants.

According to the invention, the term “CA19-9-positive cancer” means a cancer involving cancer cells expressing CA19-9, preferably on the surface of said cancer cells.

“Cell surface” is used in accordance with its normal meaning in the art, and thus includes the outside of the cell which is accessible to binding by proteins and other molecules. For example, a transmembrane protein having one or more extracellular portions is considered as being expressed on the cell surface.

CA19-9 is expressed on the surface of cells if it is located at the surface of said cells and is accessible to binding by CA19-9-specific antibodies added to the cells which have not been disrupted.

According to the invention, the term “disease” refers to any pathological state, including cancer, in particular those forms of cancer described herein. Any reference herein to cancer or particular forms of cancer also includes cancer metastasis thereof. In a preferred embodiment, a disease to be treated according to the present application involves cells expressing CA19-9.

“Diseases associated with cells expressing CA19-9” or similar expressions means according to the invention that CA19-9 is expressed in cells of a diseased tissue or organ. In one embodiment, expression of CA19-9 in cells of a diseased tissue or organ is increased compared to the state in a healthy tissue or organ. An increase refers to an increase by at least 10%, in particular at least 20%, at least 50%, at least 100%, at least 200%, at least 500%, at least 1000%, at least 10000% or even more. In one embodiment, expression is only found in a diseased tissue, while expression in a corresponding healthy tissue is repressed. For example, CA19-9 is expressed in pancreatic cancer tissue while expression is not detectable in non-cancerous pancreatic tissue. According to the invention, diseases associated with cells expressing CA19-9 include cancer diseases. Furthermore, according to the invention, cancer diseases preferably are those wherein the cancer cells express CA19-9.

As used herein, a “cancer disease” or “cancer” includes a disease characterized by aberrantly regulated cellular growth, proliferation, differentiation, adhesion, and/or migration. By “cancer cell” is meant an abnormal cell that grows by a rapid, uncontrolled cellular proliferation and continues to grow after the stimuli that initiated the new growth cease. Preferably, a “cancer disease” is characterized by cells expressing CA19-9 and a cancer cell expresses CA19-9. A cell expressing CA19-9 preferably is a cancer cell, preferably of the cancers described herein.

According to the invention, a “carcinoma” is a malignant tumor derived from epithelial cells.

“Adenocarcinoma” is a cancer that originates in glandular tissue. This tissue is also part of a larger tissue category known as epithelial tissue. Epithelial tissue includes skin, glands and a variety of other tissue that lines the cavities and organs of the body. Epithelium is derived embryologically from ectoderm, endoderm and mesoderm. To be classified as adenocarcinoma, the cells do not necessarily need to be part of a gland, as long as they have secretory properties. This form of carcinoma can occur in some higher mammals, including humans. Well differentiated adenocarcinomas tend to resemble the glandular tissue that they are derived from, while poorly differentiated may not. By staining the cells from a biopsy, a pathologist will determine whether the tumor is an adenocarcinoma or some other type of cancer. Adenocarcinomas can arise in many tissues of the body due to the ubiquitous nature of glands within the body. While each gland may not be secreting the same substance, as long as there is an exocrine function to the cell, it is considered glandular and its malignant form is therefore named adenocarcinoma. Malignant adenocarcinomas invade other tissues and often metastasize given enough time to do so.

The pancreas, an organ of endodermal derivation, is the key regulator of protein and carbohydrate digestion and glucose homeostasis. The exocrine pancreas (80% of the tissue mass of the organ) is composed of a branching network of acinar and duct cells that produce and deliver digestive enzymes into the gastrointestinal tract. The acinar cells, which are organized in functional units along the duct network, synthesize and secrete enzymes into the ductal lumen in response to cues from the stomach and duodenum. Within the acinar units near the ducts are centroacinar cells. The endocrine pancreas, which regulates metabolism and glucose homeostasis through the secretion of hormones into the bloodstream, is composed of four specialized endocrine cell types gathered together into clusters called Islets of Langerhans.

Pancreatic cancer is a malignant neoplasm originating from transformed cells arising in tissues forming the pancreas. Pancreatic cancer is the fourth most common cause of cancer-related deaths in the United States and the eighth worldwide. Early pancreatic cancer often does not cause symptoms, and the later symptoms are usually nonspecific and varied. Therefore, pancreatic cancer is often not diagnosed until it is advanced. Pancreatic cancer has a poor prognosis: for all stages combined, the 1- and 5-year relative survival rates are 25% and 6%, respectively. For local disease the 5-year survival is approximately 20% while the median survival for locally advanced and for metastatic disease, which collectively represent over 80% of individuals, is about 10 and 6 months respectively.

Pancreatic cancer includes adenocarcinomas (tumors exhibiting glandular architecture) arising within the exocrine component of the pancreas and neuroendocrine carcinomas arising from islet cells.

The most common form of pancreatic cancer, ductal adenocarcinoma, is typically characterized by moderately to poorly differentiated glandular structures on microscopic examination. Pancreatic ductal adenocarcinoma (PDAC) commonly arises in the head of the pancreas with infiltration into surrounding tissues including lymphatics, spleen, and peritoneal cavity, and with metastasis to the liver and lungs. PDAC primarily exhibits a glandular pattern with duct-like structures and varying degrees of cellular atypia and differentiation. Less common subtypes of PDAC include colloid, adenosquanous, or sarcomatoid histology. Often within an individual tumor, there are regional differences in histology, tumor grade, and degree of differentiation. Even the smallest primary lesions commonly exhibit perineural and lympho-vascular invasion, suggesting a propensity for early distant spread.

The second most common type of exocrine pancreas cancer is mucinous. Mucinous adenocarcinoma produces a large volume of mucin that results in a cystic appearance on imaging studies.

Pancreatic neuroendocrine tumors form in hormone-making cells (islet cells) of the pancreas. Acinic cell neoplasms arise from the acinar cells of the pancreas.

According to the invention, the term “cancer” also includes cancer metastasis of a primary tumor such as primary pancreatic cancer. Thus, if reference is made, for example, to pancreatic cancer, this also includes metastasis of the pancreatic cancer, for example metastasis to the lung, liver and/or lymph nodes.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential. In one embodiment, the term “metastasis” according to the invention relates to “distant metastasis” which relates to a metastasis which is remote from the primary tumor and the regional lymph node system. In one embodiment, the term “metastasis” according to the invention relates to lymph node metastasis. One particular form of metastasis which is treatable using the therapy of the invention is metastasis originating from pancreatic cancer as primary site. In preferred embodiments such pancreatic cancer metastasis is metastasis into lymph nodes, metastasis into lung and/or metastasis into liver.

A refractory cancer is a malignancy for which a particular treatment is ineffective, which is either initially unresponsive to treatment, or which becomes unresponsive over time.

By “treat” is meant to administer a compound or composition or a combination of compounds or compositions to a subject in order to prevent or eliminate a disease, including reducing the size of a tumor or the number of tumors in a subject; arrest or slow a disease in a subject; inhibit or slow the development of a new disease in a subject; decrease the frequency or severity of symptoms and/or recurrences in a subject who currently has or who previously has had a disease; and/or prolong, i.e., increase the lifespan of the subject.

In particular, the term “treatment of a disease” includes curing, shortening the duration, ameliorating, preventing, slowing down or inhibiting progression or worsening, or preventing or delaying the onset of a disease or the symptoms thereof.

The term “patient” means according to the invention a subject for treatment, in particular a diseased subject, including human beings, nonhuman primates or other animals, in particular mammals such as cows, horses, pigs, sheep, goats, dogs, cats or rodents such as mice and rats. In a particularly preferred embodiment, a patient is a human being.

The FOLFIRINOX chemotherapy regimen is described in the art (Conroy et al., 2011, N Engl J Med. 364:1817-1825). The drug combination used in FOLFIRINOX chemotherapy comprises of leucovorin, fluorouracil, irinotecan (such as irinotecan hydrochloride) and oxaliplatin. Oxaliplatin may be given at 85 mg per square meter of body-surface area; irinotecan at 180 mg per square meter; leucovorin at 400 mg per square meter; and fluorouracil at 400 mg per square meter given as a bolus followed by 5-fluorouracil at 2400 mg per square meter given as a continuous infusion of preferably 46-hours, preferably every 2 weeks).

In an embodiment, the FOLFIRINOX chemotherapy regimen comprises administration of oxaliplatin, leucovorin, irinotecan and 5-fluorouracil, and in one embodiment FOLFIRINOX can be administered at a dose of 65 mg/m² oxaliplatin, 400 mg/m² leucovorin, 150 mg/m² irinotecan, and 1200 mg/m² 5-fluorouracil. In an embodiment, FOLFIRINOX is administered intravenously at a dose of 65 mg/m² oxaliplatin on day 1, 400 mg/m² leucovorin on day 1, 150 mg/n² irinotecan on day 1, and a total of 1200 mg/m² 5-fluorouracil over days 1 and 2.

Oxaliplatin refers to a compound which is a platinum compound that is complexed to a diaminocyclohexane carrier ligand of the following formula:

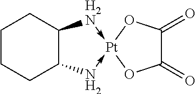

In particular, the term “oxaliplatin” refers to the compound [(1R,2R)-cyclohexane-1,2-diamine](ethanedioato-O,O′)platinum(II). Oxaliplatin for injection is also marketed under the trade name Eloxatine.

Leucovorin (folinic acid) refers to a compound useful in synergistic combination with the chemotherapy agent 5-fluorouracil. Thus, if reference is made herein to the administration of 5-fluorouracil or a prodrug thereof, said administration in one embodiment may comprise an administration in conjunction with folinic acid. Folinic acid has the following formula:

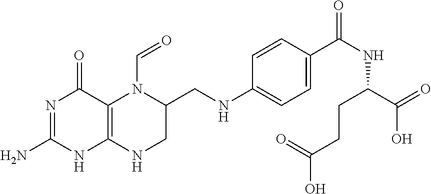 [SMILES: Nc1nc(=O)c2c([nH]1)NCC(CNc1ccc(C(=O)N[C@@H](CCC(=O)O)C(=O)O)cc1)N2C=O]

In particular, the term refers to the compound (2S)-2-{[4-[(2-amino-5-formyl-4-oxo-5,6,7,8-tetrahydro-1H-pteridin-6-yl)methylamino]benzoyl]amino}pentanedioic acid.

Irinotecan is a drug preventing DNA from unwinding by inhibition of topoisomerase I. In chemical terms, it is a semisynthetic analogue of the natural alkaloid camptothecin having the following formula:

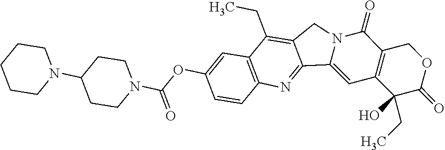 [SMILES: CCc1c2c(nc3ccc(OC(=O)N4CCC(N5CCCCC5)CC4)cc13)-c1cc3c(c(=O)n1C2)COC(=O)[C@]3(O)CC]

In particular, the term “irinotecan” refers to the compound (S)-4,11-diethyl-3,4,12,14-tetrahydro-4-hydroxy-3,14-dioxo1H-pyrano[3′,4′:6,7]-indolizino[1,2-b]quinolin-9-yl-[1,4′-bipiperidine]-1′-carboxylate.

Fluorouracil or 5-fluorouracil (5-FU or f5U) (sold under the brand names Adrucil, Carac, Efudix, Efudex and Fluoroplex) is a compound which is a pyrimidine analog of the following formula:

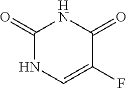

In particular, the term refers to the compound 5-fluoro-1H-pyrimidine-2,4-dione.

According to the invention, the patient can be administered, in addition to the anti-CA19-9 antibody and FOLFIRINOX, other chemotherapeutic agents or combinations of chemotherapeutic agents such as cytostatic agents. Chemotherapeutic agents may affect cells in one of the following ways: (1) damage the DNA of the cells so they can no longer reproduce, (2) inhibit the synthesis of new DNA strands so that no cell replication is possible, (3) stop the mitotic processes of the cells so that the cells cannot divide into two cells.

The activity of a cytostatic compound or a combination of cytostatic compounds results in the cells being arrested in or accumulating in one or more phases of the cell cycle, preferably in one or more phases of the cell cycle other than the G1- and G0-phases, preferably other than the G1-phase, preferably in one or more of the G2- or S-phase of the cell cycle such as the G1/G2-, S/G2-, G2- or S-phase of the cell cycle. The term “cells being arrested in or accumulating in one or more phases of the cell cycle” means that the percentage of cells which are in said one or more phases of the cell cycle increases. Each cell goes through a cycle comprising four phases in order to replicate itself. The first phase called G1 is when the cell prepares to replicate its chromosomes. The second stage is called S, and in this phase DNA synthesis occurs and the DNA is duplicated. The next phase is the G2 phase, when the RNA and protein duplicate. The final stage is the M stage, which is the stage of actual cell division. In this final stage, the duplicated DNA and RNA split and move to separate ends of the cell, and the cell actually divides into two identical, functional cells. Chemotherapeutic agents which are DNA damaging agents usually result in an accumulation of cells in the G1 and/or G2 phase. Chemotherapeutic agents which block cell growth by interfering with DNA synthesis such as antimetabolites usually result in an accumulation of cells in the S-phase. Examples of these drugs are gemcitabine and 6-mercaptopurine.

According to the invention, other chemotherapeutic agents can include nucleoside analogs such as gemcitabine or prodrugs thereof, platinum compounds such as cisplatin, taxanes such as paclitaxel and docetaxel, and camptothecin analogs such as topotecan, and combinations of drugs such as combinations of drugs, and includes any prodrug such as ester, salt or derivative such as conjugate of said agent. Examples are conjugates of said agent with a carrier substance, e.g., protein-bound paclitaxel such as albumin-bound paclitaxel. Preferably, salts of said agent are pharmaceutically acceptable.

In specific circumstances, cancer cells can enter a lethal stress pathway linked to the emission of a spatiotemporally defined combination of signals that is decoded by the immune system to activate tumor-specific immune responses (Zitvogel et al., 2010 Cell 140:798-804). In such scenario, cancer cells are triggered to emit signals that are sensed by innate immune effectors such as dendritic cells to trigger a cognate immune response that involves CD8+ T cells and IFN-γ signaling so that tumor cell death may elicit a productive anticancer immune response. These signals include the pre-apoptotic exposure of the endoplasmic reticulum (ER) chaperon calreticulin (CRT) at the cell surface, the pre-apoptotic secretion of ATP, and the post-apoptotic release of the nuclear protein HMGB1. Together, these processes constitute the molecular determinants of immunogenic cell death (ICD). Anthracyclines, oxaliplatin, and γ irradiation are able to induce all signals that define ICD, while cisplatin, for example, which is deficient in inducing CRT translocation from the ER to the surface of dying cells—a process requiring ER stress—requires complementation by thapsigargin, an ER stress inducer.

According to the invention, other chemotherapeutic agents includes an agent or a combination of agents which when provided to cells, in particular cancer cells, is capable of inducing the cells to enter a lethal stress pathway which finally results in tumor-specific immune responses. In particular, an agent inducing immunogenic cell death when provided to cells induces the cells to emit a spatiotemporally defined combination of signals, including, in particular, the pre-apoptotic exposure of the endoplasmic reticulum (ER) chaperon calreticulin (CRT) at the cell surface, the pre-apoptotic secretion of ATP, and the post-apoptotic release of the nuclear protein HMGB1. Exemplary agents include anthracyclines. Anthracyclines are a class of drugs commonly used in cancer chemotherapy that are also antibiotics. Structurally, all anthracyclines share a common four-ringed 7,8,9,10-tetrahydrotetracene-5,12-quinone structure and usually require glycosylation at specific sites.

Anthracyclines preferably bring about one or more of the following mechanisms of action: 1. Inhibiting DNA and RNA synthesis by intercalating between base pairs of the DNA/RNA strand, thus preventing the replication of rapidly-growing cancer cells. 2. Inhibiting topoisomerase II enzyme, preventing the relaxing of supercoiled DNA and thus blocking DNA transcription and replication. 3. Creating iron-mediated free oxygen radicals that damage the DNA and cell membranes.

Examples of anthracyclines and anthracycline analogs include, but are not limited to, daunorubicin (daunomycin), doxorubicin (adriamycin), epirubicin, idarubicin, rhodomycin, pyrarubicin, valrubicin, N-trifluoro-acetyl doxorubicin-14-valerate, aclacinomycin, morpholinodoxorubicin (morpholino-DOX), cyanomorpholino-doxorubicin (cyano-morpholino-DOX), 2-pyrrolino-doxorubicin (2-PDOX), 5-iminodaunomycin, mitoxantrone and aclacinomycin A (aclarubicin). Mitoxantrone is a member of the anthracendione class of compounds, which are anthracycline analogs that lack the sugar moiety of the anthracyclines but retain the planar polycylic aromatic ring structure that permits intercalation into DNA.

Specifically contemplated as anthracycline in the context of the present invention is epirubicin, which is marketed under the trade name Ellence in the US and Pharmorubicin or Epirubicin Ebewe elsewhere. In particular, the term “epirubicin” refers to the compound (8R,10S)-10-[(2S,4S,5R,6S)-4-amino-5-hydroxy-6-methyl-oxan-2-yl]oxy-6,11-dihydroxy-8-(2-hydroxyacetyl)-1-methoxy-8-methyl-9,10-dihydro-7H-tetracen-5,12-dion. Epirubicin is favored over doxorubicin, the most popular anthracycline, in some chemotherapy regimens as it appears to cause fewer side-effects.

Other chemotherapeutic agents include nucleoside analogs, which are structural analogs of a nucleoside, a category that includes both purine analogs and pyrimidine analogs, such as gemcitabine and capecitabine. Capecitabine (Xeloda, Roche) refers to a chemotherapeutic agent that is a prodrug that is converted into 5-FU in the tissues. http://upload.wikimedia.org/wikipedia/commons/b/bO/Capecitabine-from-xtal-2009-2D-skeletal.png

Other chemotherapeutic agents include platinum compounds, which are compounds containing platinum in their structure such as platinum complexes and includes compounds such as cisplatin and carboplatin.

Other chemotherapeutic agents include taxane compounds. Taxanes are a class of diterpene compounds that were first derived from natural sources such as plants of the genus Taxus, but some have been synthesized artificially. The principal mechanism of action of the taxane class of drugs is the disruption of microtubule function, thereby inhibiting the process of cell division. Taxanes include docetaxel (Taxotere) and paclitaxel (Taxol).

Other chemotherapeutic agents include camptothecin analogs. According to the invention, preferred camptothecin analogs are inhibitors of DNA enzyme topoisomerase I (topo 1). A preferred camptothecin analog is topotecan.

γδ T cells (gamma delta T cells) represent a small subset of T cells that possess a distinct T cell receptor (TCR) on their surface. A majority of T cells have a TCR composed of two glycoprotein chains called α- and β-TCR chains. In contrast, in γδ T cells, the TCR is made up of one γ-chain and one δ-chain. This group of T cells is usually much less common than αβ T cells. Human γδ T cells play an important role in stress-surveillance responses like infectious diseases and autoimmunity. Transformation-induced changes in tumors are also suggested to cause stress-surveillance responses mediated by γδ T cells and enhance antitumor immunity. Importantly, after antigen engagement, activated γδ T cells at lesional sites provide cytokines (e.g., INFγ, TNFα) and/or chemokines mediating recruitment of other effector cells and show immediate effector functions such as cytotoxicity (via death receptor and cytolytic granules pathways) and ADCC.

The majority of γδ T cells in peripheral blood express the Vγ9Vδ2 T cell receptor (TCRγδ). Vγ9Vδ2 T cells are unique to humans and primates and are assumed to play an early and essential role in sensing “danger” by invading pathogens as they expand dramatically in many acute infections and may exceed all other lymphocytes within a few days, e.g., in tuberculosis, salmonellosis, ehrlichiosis, brucellosis, tularemia, listeriosis, toxoplasmosis, and malaria.

γδ T cells respond to small non-peptidic phosphorylated antigens (phosphoantigens) such as pyrophosphates synthesized in bacteria and isopentenyl pyrophosphate (IPP) produced in mammalian cells through the mevalonate pathway. Whereas IPP production in normal cells is not sufficient for activation of 78 T cells, dysregulation of the mevalonate pathway in tumor cells leads to accumulation of IPP and γδ T cell activation. IPPs can also be therapeutically increased by aminobisphosphonates, which inhibit the mevalonate pathway enzyme farnesyl pyrophosphate synthase (FPPS). Among others, zoledronic acid (ZA, zoledronate, Zometa™, Novartis) represents such an aminobiphosphonate, which is already clinically administered to patients for the treatment of osteoporosis and metastasic bone disease. Upon treatment of PBMCs in vitro, ZA is taken up especially by monocytes. IPP accumulates in the monocytes and they differentiate to antigen-presenting cells stimulating development of γδ T cells. In this setting, the addition of interleukin-2 (IL-2) is preferred as growth and survival factor for activated YS T cells. Finally, certain alkylated amines have been described to activate Vγ9Vδ2 T cells in vitro, however only at millimolar concentrations.

According to the invention, the term “agent stimulating γδ T cells” relates to compounds stimulating development of 78 T cells, in particular Vγ9Vδ2 T cells, in vitro and/or in vivo, in particular by inducing activation and expansion of γδ T cells. Preferably, the term relates to compounds which in vitro and/or in vivo increase isopentenyl pyrophosphate (IPP) produced in mammalian cells, preferably by inhibiting the mevalonate pathway enzyme farnesyl pyrophosphate synthase (FPPS).

One particular group of compounds stimulating γδ T cells are bisphosphonates, in particular nitrogen-containing bisphosphonates (N-bisphosphonates; aminobisphosphonates).

For example, suitable bisphosphonates for use in the invention may include one or more of the following compounds including analogs and derivatives, pharmaceutical salts, hydrates, esters, conjugates and prodrugs thereof:
[1-hydroxy-2-(1H-imidazol-1-yl)ethane-1,1-diyl]bis(phosphonic acid), zoledronic acid, e.g. zoledronate;
(dichloro-phosphono-methyl)phosphonic acid, e.g. clodronate
{1-hydroxy-3-[methyl(pentyl)amino]propane-1,1-diyl}bis(phosphonic acid), ibandronic acid, e.g. ibandronate
(3-amino-1-hydroxypropane-1,1-diyl)bis (phosphonic acid), pamidronic acid, e.g. pamidronate;
(1-hydroxy-1-phosphono-2-pyridin-3-yl-ethyl)phosphonic acid, risedronic acid, e.g. risedronate;
(1-Hydroxy-2-imidazo[1,2-a]pyridin-3-yl-1-phosphonoethyl)phosphonic acid, minodronic acid;
[3-(dimethylamino)-1-hydroxypropane-1,1-diyl]bis(phosphonic acid), olpadronic acid;
[4-amino-1-hydroxy-1-(hydroxy-oxido-phosphoryl)-butyl]phosphonic acid, alendronic acid, e.g. alendronate;
[(Cycloheptylamino)methylene]bis(phosphonic acid), incadronic acid;
(1-hydroxyethan-1,1-diyl)bis(phosphonic acid), etidronic acid, e.g. etidronate; and
{[(4-chlorophenyl)thio]methylene}bis(phosphonic acid), tiludronic acid.

According to the invention, zoledronic acid (INN) or zoledronate (marketed by Novartis under the trade names Zometa, Zomera, Aclasta and Reclast) is a particularly preferred bisphosphonate. Zometa is used to prevent skeletal fractures in patients with cancers such as multiple myeloma and prostate cancer, as well as for treating osteoporosis. It can also be used to treat hypercalcemia of malignancy and can be helpful for treating pain from bone metastases.

In one particularly preferred embodiment, an agent stimulating γδ T cells according to the invention is administered in combination with IL-2. Such combination has been shown to be particularly effective in mediating expansion and activation of γ9δ2 T cells.

Interleukin-2 (IL-2) is an interleukin, a type of cytokine signaling molecule in the immune system. It is a protein that attracts lymphocytes and is part of the body's natural response to microbial infection, and in discriminating between foreign (non-self) and self. IL-2 mediates its effects by binding to IL-2 receptors, which are expressed by lymphocytes.

The IL-2 used according to the invention may be any IL-2 supporting or enabling the stimulation of γδ T cells and may be derived from any species, preferably human. Il-2 may be isolated, recombinantly produced or synthetic IL-2 and may be naturally occurring or modified IL-2.

The term “antigen” relates to an agent such as a protein or peptide comprising an epitope against which an immune response is directed and/or is to be directed. In a preferred embodiment, an antigen is a tumor-associated antigen, such as CA19-9, i.e., a constituent of cancer cells which may be derived from the cytoplasm, the cell surface and the cell nucleus, in particular those antigens which are produced, preferably in large quantity, intracellular or as surface antigens on cancer cells.

In the context of the present invention, the term “tumor-associated antigen” preferably relates to proteins that are under normal conditions specifically expressed in a limited number of tissues and/or organs or in specific developmental stages and are expressed or aberrantly expressed in one or more tumor or cancer tissues. In the context of the present invention, the tumor-associated antigen is preferably associated with the cell surface of a cancer cell and is preferably not or only rarely expressed in normal tissues.

The term “epitope” refers to an antigenic determinant in a molecule, i.e., to the part in a molecule that is recognized by the immune system, for example, that is recognized by an antibody. For example, epitopes are the discrete, three-dimensional sites on an antigen, which are recognized by the immune system. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. A preferred epitope of CA19-9 is the Sialyl Lewis A (sLe^{a}) antigen epitope.

As used in the context of the present invention, the term “antibody having the ability to bind to CA19-9” encompasses any molecule or agent which has the ability to bind to CA 19-9, and in particular includes molecules or agents that comprise the antigen binding domain of an antibody having the ability to bind CA 19-9. For example, encompassed within this term is a molecule that comprises the heavy and light chain variable regions of any antibody having the ability to bind to CA 19-9, including any of the particular monoclonal antibodies described herein.

The term “antibody” refers to a glycoprotein comprising at least two heavy (H) chains and two light (L) chains inter-connected by disulfide bonds, and includes any molecule comprising an antigen binding portion thereof. The term “antibody” includes monoclonal antibodies and fragments or derivatives of antibodies, including, without limitation, human antibodies, humanized antibodies, chimeric antibodies, single chain antibodies, e.g., scFv's and antigen-binding antibody fragments such as Fab and Fab′ fragments and also includes all recombinant forms of antibodies, e.g., antibodies expressed in prokaryotes, unglycosylated antibodies, and any antigen-binding antibody fragments and derivatives as described herein. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The antibodies described herein may be human antibodies. The term “human antibody”, as used herein, is intended to include antibodies having variable and constant regions derived from human germline immunoglobulin sequences. The human antibodies described herein may include amino acid residues not encoded by human germline immunoglobulin sequences (e.g., mutations introduced by random or site-specific mutagenesis in vitro or by somatic mutation in vivo).

The term “humanized antibody” refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen binding sites may be wild-type or modified by one or more amino acid substitutions, e.g. modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

The term “chimeric antibody” refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another.

Typically, the variable region of both light and heavy chains mimics the variable regions of antibodies derived from one species of mammals, while the constant portions are homologous to sequences of antibodies derived from another. One clear advantage to such chimeric forms is that the variable region can conveniently be derived from presently known sources using readily available B-cells or hybridomas from non-human host organisms in combination with constant regions derived from, for example, human cell preparations. While the variable region has the advantage of ease of preparation and the specificity is not affected by the source, the constant region being human, is less likely to elicit an immune response from a human subject when the antibodies are injected than would the constant region from a non-human source. However, the definition is not limited to this particular example.

The terms “antigen-binding portion” of an antibody (or simply “binding portion”) or “antigen-binding fragment” of an antibody (or simply “binding fragment”) or similar terms refer to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) Fab fragments, monovalent fragments consisting of the VL, VH, CL and CH domains; (ii) F(ab′)₂ fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., 1989, Nature 341:544-546), which consist of a VH domain; (vi) isolated complementarity determining regions (CDR), and (vii) combinations of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., 1988, Science 242:423-426; and Huston et al., 1988, Proc. Natl. Acad. Sci. USA 85:5879-5883). Such single chain antibodies are also intended to be encompassed within the term “antigen-binding fragment” of an antibody. A further example is binding-domain immunoglobulin fusion proteins comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. Binding-domain immunoglobulin fusion proteins are further disclosed in U.S. Patent Application Publication Nos. 2003/0118592 and 2003/0133939. These antibody fragments are obtained using conventional techniques known to those with skill in the art, and the fragments are screened for utility in the same manner as are intact antibodies.

The term “bispecific molecule” is intended to include any agent, e.g., a protein, peptide, or protein or peptide complex, which has two different binding specificities. For example, the molecule may bind to, or interact with (a) a cell surface antigen, and (b) an Fc receptor on the surface of an effector cell. The term “multispecific molecule” or “heterospecific molecule” is intended to include any agent, e.g., a protein, peptide, or protein or peptide complex, which has more than two different binding specificities. For example, the molecule may bind to, or interact with (a) a cell surface antigen, (b) an Fe receptor on the surface of an effector cell, and (c) at least one other component. Accordingly, the invention includes, but is not limited to, bispecific, trispecific, tetraspecific, and other multispecific molecules which are directed to CA19-9, and to other targets, such as Fe receptors on effector cells. The term “bispecific antibodies” also includes diabodies. Diabodies are bivalent, bispecific antibodies in which the VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger et al., 1993, Proc. Natl. Acad. Sci. USA 90:6444-6448; Poljak et al., 1994, Structure 2:1121-1123).

In certain embodiments, the antibody is conjugated to a therapeutic moiety or agent, such as a cytotoxin, a drug (e.g., an immunosuppressant) or a radioisotope. A cytotoxin or cytotoxic agent includes any agent that is detrimental to and, in particular, kills cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Suitable therapeutic agents for forming antibody conjugates include, but are not limited to, antimetabolites (e.g., methotrexate, 6-mercaptopurine, 6-thioguanine, cytarabine, fludarabin, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC), and anti-mitotic agents (e.g., vincristine and vinblastine). In a preferred embodiment, the therapeutic agent is a cytotoxic agent or a radiotoxic agent. In another embodiment, the therapeutic agent is an immunosuppressant. In yet another embodiment, the therapeutic agent is GM-CSF. In a preferred embodiment, the therapeutic agent is doxorubicin, cisplatin, bleomycin, sulfate, carmustine, chlorambucil, cyclophosphamide or ricin A.

Antibodies also can be conjugated to a radioisotope, e.g., iodine-131, yttrium-90 or indium-111, to generate cytotoxic radiopharmaceuticals.

Antibody conjugates of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, Pseudomonas exotoxin, or diphtheria toxin; a protein such as tumor necrosis factor or interferon-γ; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Techniques for conjugating such therapeutic moiety to antibodies are well known, see, e.g., Arnon et al., Monoclonal Antibodies For Immunotargeting Of Drugs In Cancer Therapy, in Monoclonal Antibodies And Cancer Therapy, Reisfeld et al. (eds.), pp. 243-56 (Alan R. Liss, Inc. 1985); Hellstrom et al., Antibodies For Drug Delivery, in Controlled Drug Delivery (2nd Ed.), Robinson et al. (eds.), pp. 623-53 (Marcel Dekker, Inc. 1987); Thorpe, Antibody Carriers Of Cytotoxic Agents In Cancer Therapy: A Review, in Monoclonal Antibodies '84: Biological And Clinical Applications, Pinchera et al. (eds.), pp. 475-506 (1985); Analysis, Results, And Future Prospective Of The Therapeutic Use Of Radiolabeled Antibody In Cancer Therapy, in Monoclonal Antibodies For Cancer Detection And Therapy, Baldwin et al. (eds.), pp. 303-16 (Academic Press 1985), and Thorpe et al., The Preparation And Cytotoxic Properties Of Antibody-Toxin Conjugates, Immunol. Rev., 62: 119-58 (1982).

As used herein, an antibody is “derived from” a particular germline sequence if the antibody is obtained from a system by immunizing an animal or by screening an immunoglobulin gene library, and wherein the selected antibody is at least 90%, more preferably at least 95%, even more preferably at least 96%, 97%, 98%, or 99% identical in amino acid sequence to the amino acid sequence encoded by the germline immunoglobulin gene. Typically, an antibody derived from a particular germline sequence will display no more than 10 amino acid differences, more preferably, no more than 5, or even more preferably, no more than 4, 3, 2, or 1 amino acid difference from the amino acid sequence encoded by the germline immunoglobulin gene.

As used herein, the term “heteroantibodies” refers to two or more antibodies, derivatives thereof, or antigen binding regions linked together, at least two of which have different specificities. These different specificities include a binding specificity for an Fe receptor on an effector cell, and a binding specificity for an antigen or epitope on a target cell, e.g., a tumor cell.

The antibodies described herein may be monoclonal antibodies. The term “monoclonal antibody” as used herein refers to a preparation of antibody molecules of single molecular composition. A monoclonal antibody displays a single binding specificity and affinity. In one embodiment, the monoclonal antibodies are produced by a hybridoma which includes a B cell obtained from a non-human animal, e.g., mouse, fused to an immortalized cell.

The antibodies described herein may be recombinant antibodies. The term “recombinant antibody”, as used herein, includes all antibodies that are prepared, expressed, created or isolated by recombinant means, such as (a) antibodies isolated from an animal (e.g., a mouse) that is transgenic or transchromosomal with respect to the immunoglobulin genes or a hybridoma prepared therefrom, (b) antibodies isolated from a host cell transformed to express the antibody, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences.

Antibodies described herein may be derived from different species, including but not limited to mouse, rat, rabbit, guinea pig and human.

Antibodies described herein include polyclonal and monoclonal antibodies and include IgA such as IgA1 or IgA2, IgG1, IgG2, IgG3, IgG4, IgE, IgM, and IgD antibodies. In various embodiments, the antibody is an IgG1 antibody, more particularly an IgG1, kappa or IgG1, lambda isotype (i.e. IgG1, κ, λ), an IgG2a antibody (e.g. IgG2a, κ, λ), an IgG2b antibody (e.g. IgG2b, κ, λ), an IgG3 antibody (e.g. IgG3, κ, λ) or an IgG4 antibody (e.g. IgG4, κ, λ).

The term “transfectoma”, as used herein, includes recombinant eukaryotic host cells expressing an antibody, such as CHO cells, NS/0 cells, HEK293 cells, HEK293T cells, plant cells, or fungi, including yeast cells.

As used herein, a “heterologous antibody” is defined in relation to a transgenic organism producing such an antibody. This term refers to an antibody having an amino acid sequence or an encoding nucleic acid sequence corresponding to that found in an organism not consisting of the transgenic organism, and being generally derived from a species other than the transgenic organism.

As used herein, a “heterohybrid antibody” refers to an antibody having light and heavy chains of different organismal origins. For example, an antibody having a human heavy chain associated with a murine light chain is a heterohybrid antibody.

The invention includes all antibodies and derivatives of antibodies as described herein which for the purposes of the invention are encompassed by the term “antibody”. The term “antibody derivatives” refers to any modified form of an antibody, e.g., a conjugate of the antibody and another agent or antibody, or an antibody fragment.

Antibodies described herein are preferably isolated. An “isolated antibody” as used herein, is intended to refer to an antibody which is substantially free of other antibodies having different antigenic specificities (e.g., an isolated antibody that specifically binds to CA19-9 is substantially free of antibodies that specifically bind antigens other than CA19-9). An isolated antibody that specifically binds to an epitope, isoform or variant of human CA19-9 may, however, have cross-reactivity to other related antigens, e.g., from other species (e.g., CA19-9 species homologs). Moreover, an isolated antibody may be substantially free of other cellular material and/or chemicals. In one embodiment of the invention, a combination of “isolated” monoclonal antibodies relates to antibodies having different specificities and being combined in a well-defined composition or mixture.

A CDR refers to one of three hypervariable regions (H1, H2 or H3) within the non-framework region of the immunoglobulin (Ig or antibody) VH j-sheet framework, or one of three hypervariable regions (L1, L2 or L3) within the non-framework region of the antibody VL 0-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., 1977, J. Biol. Chem. 252:6609-6616; Kabat, 1978, Adv. Prot. Chem. 32:1-75). CDR region sequences also have been defined structurally by Chothia as those residues that are not part of the conserved 1-sheet framework, and thus are able to adapt different conformations (Chothia and Lesk, 1987, J. Mol. Biol. 196:901-917). Both terminologies are well recognized in the art. The positions of CDRs within a canonical antibody variable domain have been determined by comparison of numerous structures (Al-Lazikani et al., 1997, J. Mol. Biol. 273:927-948; Morea et al., 2000, Methods 20:267-279). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra). Such nomenclature is similarly well known to those skilled in the art.

For example, CDRs defined according to either the Kabat (hypervariable) or Chothia (structural) designations, are set forth in the Table 1 below.

TABLE 1
CDR Definitions

| | Kabat¹ | Chothia² | Loop Location |
|---|---|---|---|
| VH CDR1 | 31-35 | 26-32 | linking B and C strands |
| VH CDR2 | 50-65 | 53-55 | linking C′ and C″ strands |
| VH CDR3 | 95-102 | 96-101 | linking F and G strands |
| VL CDR1 | 24-34 | 26-32 | linking B and C strands |
| VL CDR2 | 50-56 | 50-52 | linking C′ and C″ strands |
| VL CDR3 | 89-97 | 91-96 | linking F and G strands |

¹Residue numbering follows the nomenclature of Kabat et al., supra;
²Residue numbering follows the nomenclature of Chothia et al., supra.

One or more CDRs also can be incorporated into a molecule either covalently or noncovalently to make it an immunoadhesin. An immunoadhesin can incorporate the CDR(s) as part of a larger polypeptide chain, can co-valently link the CDR(s) to another polypeptide chain, or can incorporate the CDR(s) noncovalently. The CDRs permit the immunoadhesin to bind to a particular antigen of interest.

The term “binding” according to the invention preferably relates to a specific binding.

According to the present invention, an antibody is capable of binding to a predetermined target if it has a significant affinity for said predetermined target and binds to said predetermined target in standard assays. “Affinity” or “binding affinity” is often measured by equilibrium dissociation constant (K_{D}). Preferably, the term “significant affinity” refers to the binding to a predetermined target with a dissociation constant (K_{D}) of 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, or 10⁻¹² M or lower.

An antibody is not (substantially) capable of binding to a target if it has no significant affinity for said target and does not bind significantly, in particular does not bind detectably, to said target in standard assays. Preferably, the antibody does not detectably bind to said target if present in a concentration of up to 2, preferably 10, more preferably 20, in particular 50 or 100 μg/ml or higher. Preferably, an antibody has no significant affinity for a target if it binds to said target with a K_{D} that is at least 10-fold, 100-fold, 10³-fold, 10⁴-fold, 10⁵-fold, or 10⁶-fold higher than the K_{D} for binding to the predetermined target to which the antibody is capable of binding. For example, if the K_{D} for binding of an antibody to the target to which the antibody is capable of binding is 10⁻⁷ M, the K_{D} for binding to a target for which the antibody has no significant affinity would be is at least 10⁻⁶ M, 10⁻⁵ M, 10⁻⁴ M, 10⁻³ M, 10⁻² M, or 10⁻¹ M.

An antibody is specific for a predetermined target if it is capable of binding to said predetermined target while it is not capable of binding to other targets, i.e. has no significant affinity for other targets and does not significantly bind to other targets in standard assays. According to the invention, an antibody is specific for CA19-9 if it is capable of binding to CA19-9 but is not (substantially) capable of binding to other targets. Preferably, an antibody is specific for CA19-9 if the affinity for and the binding to such other targets does not significantly exceed the affinity for or binding to CA19-9-unrelated proteins such as bovine serum albumin (BSA), casein, human serum albumin (HSA) or transmembrane proteins such as MHC molecules or transferrin receptor or any other specified polypeptide. Preferably, an antibody is specific for a predetermined target if it binds to said target with a K_{D} that is at least 10-fold, 100-fold, 10³-fold, 10⁴-fold, 10⁵-fold, or 10⁶-fold lower than the K_{D} for binding to a target for which it is not specific. For example, if the K_{D} for binding of an antibody to the target for which it is specific is 10⁻⁷ M, the K_{D} for binding to a target for which it is not specific would be at least 10⁻⁶ M, 10⁻⁵ M, 10⁻⁴ M, 10⁻³ M, 10⁻² M, or 10⁻¹ M.

Binding of an antibody to a target can be determined experimentally using any suitable method; see, for example, Berzofsky et al., Antibody-Antigen Interactions In Fundamental Immunology, Paul, W. E., Ed., Raven Press New York, N Y (1984), Kuby, Janis Immunology, W. H. Freeman and Company New York, N Y (1992), and methods described herein. Affinities may be readily determined using conventional techniques, such as by equilibrium dialysis; by using the BIAcore 2000 instrument, using general procedures outlined by the manufacturer; by radioimmunoassay using radiolabeled target antigen; or by another method known to the skilled artisan. The affinity data may be analyzed, for example, by the method of Scatchard et al., Ann N.Y. Acad. ScL, 51:660 (1949). The measured affinity of a particular antibody-antigen interaction can vary if measured under different conditions, e.g., salt concentration, pH. Thus, measurements of affinity and other antigen-binding parameters, e.g., K_{D}, IC₅₀, are preferably made with standardized solutions of antibody and antigen, and a standardized buffer.

The strength of the total non-covalent interactions between a single antigen-binding site on an antibody or functional fragment and a single epitope of a target molecule, such as sLe^{a}, is the affinity of the antibody or functional fragment for that epitope. he ratio of association (k1) to dissociation (k−1) of an antibody or functional fragment thereof to a monovalent antigen (k1/k−1) is the association constant K, which is a measure of affinity. The value of K varies for different complexes of antibody or functional fragment and antigen and depends on both k1 and k−1. The association constant K for an antibody or functional fragment of the invention can be determined using any method provided herein or any other method well known to those skilled in the art.

The affinity at one binding site does not always reflect the true strength of the interaction between an antibody or functional fragment and an antigen. When complex antigens containing multiple, repeating antigenic determinants, such as a polyvalent sLe^{a}, come in contact with antibodies containing multiple binding sites, the interaction of antibody or functional fragment with antigen at one site will increase the probability of a reaction at a second site. The strength of such multiple interactions between a multivalent antibody and antigen is called the avidity. The avidity of an antibody or functional fragment can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

The specificity of an antibody or functional fragment thereof refers to the ability of an individual antibody or functional fragment thereof to react with only one antigen. An antibody or functional fragment can be considered specific when it can distinguish differences in the primary, secondary or tertiary structure of an antigen or isomeric forms of an antigen.

As used herein, “isotype” refers to the antibody class (e.g., IgM or IgG1) that is encoded by heavy chain constant region genes.

As used herein, “isotype switching” refers to the phenomenon by which the class, or isotype, of an antibody changes from one Ig class to one of the other Ig classes.

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally occurring.

The term “rearranged” as used herein refers to a configuration of a heavy chain or light chain immunoglobulin locus wherein a V segment is positioned immediately adjacent to a D-J or J segment in a conformation encoding essentially a complete VH or VL domain, respectively. A rearranged immunoglobulin (antibody) gene locus can be identified by comparison to germline DNA; a rearranged locus will have at least one recombined heptamer/nonamer homology element.

The term “unrearranged” or “germline configuration” as used herein in reference to a V segment refers to the configuration wherein the V segment is not recombined so as to be immediately adjacent to a D or J segment.

According to the invention an antibody having the ability of binding to CA19-9 is an antibody capable of binding to an epitope present in CA19-9, preferably the Sialyl Lewis A (sLe^{a}) antigen epitope present on CA 19-9. According to the invention an antibody having the ability of binding to CA19-9 binds to native epitopes of CA19-9 present on the surface of living cells. An antibody having the ability to bind to CA19-9 can be obtained by a method comprising the step of immunizing an animal with CA19-9 or a cell expressing CA19-9 on the cell surface. The antibody binds to cancer cells, in particular cells of the cancer types mentioned above and, preferably, does not bind substantially to non-cancerous cells.

Preferably, binding of an antibody having the ability to bind to CA19-9 to cells expressing CA19-9 induces or mediates killing of cells expressing CA19-9. The cells expressing CA19-9 are preferably cancer cells and are, in particular, selected from the group consisting of tumorigenic gastric, esophageal, pancreatic, lung, ovarian, colon, hepatic, head-neck, and gallbladder cancer cells. Preferably, the antibody induces or mediates killing of cells by inducing one or more of complement dependent cytotoxicity (CDC) mediated lysis, antibody dependent cellular cytotoxicity (ADCC) mediated lysis, apoptosis, and inhibition of proliferation of cells expressing CA19-9. Preferably, ADCC mediated lysis of cells takes place in the presence of effector cells, which in particular embodiments are selected from the group consisting of monocytes, mononuclear cells, NK cells and PMNs. Inhibiting proliferation of cells can be measured in vitro by determining proliferation of cells in an assay using bromodeoxyuridine (5-bromo-2-deoxyuridine, BrdU). BrdU is a synthetic nucleoside which is an analogue of thymidine and can be incorporated into the newly synthesized DNA of replicating cells (during the S phase of the cell cycle), substituting for thymidine during DNA replication. Detecting the incorporated chemical using, for example, antibodies specific for BrdU indicates cells that were actively replicating their DNA.

In preferred embodiments, antibodies described herein can be characterized by one or more of the following properties:
a) specificity for CA19-9;
b) a binding affinity to CA19-9 of about 100 nM or less, preferably, about 5-10 nM or less and, more preferably, about 1-3 nM or less,
c) the ability to induce or mediate CDC on CA19-9 positive cells;
d) the ability to induce or mediate ADCC on CA19-9 positive cells;
e) the ability to inhibit the growth of CA19-9 positive cells;
f) the ability to induce apoptosis of CA19-9 positive cells.

In a particularly preferred embodiment, an antibody having the ability of binding to CA19-9 is described in International Patent Application Publication No. WO 2015/053871, which is incorporated herein in its entirety.

In certain embodiments, antibodies, in particular chimerized forms of antibodies according to the invention include antibodies comprising a heavy chain constant region (CH) comprising an amino acid sequence derived from a human heavy chain constant region such as the amino acid sequence represented by SEQ ID NO: 2 or a fragment thereof. In further preferred embodiments, antibodies, in particular chimerized forms of antibodies according to the invention include antibodies comprising a light chain constant region (CL) comprising an amino acid sequence derived from a human light chain constant region such as the amino acid sequence represented by SEQ ID NO: 4 or a fragment thereof. In a particular preferred embodiment, antibodies, in particular chimerized forms of antibodies according to the invention include antibodies which comprise a CH comprising an amino acid sequence derived from a human CH such as the amino acid sequence represented by SEQ ID NO: 2 or a fragment thereof and which comprise a CL comprising an amino acid sequence derived from a human CL such as the amino acid sequence represented by SEQ ID NO: 4 or a fragment thereof.

“Fragment” or “fragment of an amino acid sequence” as used above relates to a part of an antibody sequence, i.e., a sequence which represents the antibody sequence shortened at the N- and/or C-terminus, which when it replaces said antibody sequence in an antibody retains binding of said antibody to CA19-9 and preferably functions of said antibody as described herein, e.g. CDC mediated lysis or ADCC mediated lysis. Preferably, a fragment of an amino acid sequence comprises at least 80%, preferably at least 90%, 95%, 96%, 97%, 98%, or 99% of the amino acid residues from said amino acid sequence. A fragment of an amino acid sequence selected from the group consisting of SEQ ID NOs: 2, 4, 6, 8, 10, 12, 14, and 16, preferably relates to said sequence wherein 17, 18, 19, 20, 21, 22 or 23 amino acids at the N-terminus are removed.

In a preferred embodiment, an antibody having the ability of binding to CA19-9 comprises a heavy chain variable region (VH) comprising an amino acid sequence selected from the group consisting of SEQ ID NOs: 2, 6, 10, 14, and a fragment thereof.

In a preferred embodiment, an antibody having the ability of binding to CA19-9 comprises a light chain variable region (VL) comprising an amino acid sequence selected from the group consisting of SEQ ID NO: 3, 7, 11, 15, and a fragment thereof.

Accordingly, the antibody or functional fragment thereof having the ability to bind to CA19-9 includes a VH domain that has an amino acid sequence selected from the group consisting of residues 20-142 of SEQ ID NO: 2, residues 20-142 of SEQ ID NO: 6, residues 20-142 of SEQ ID NO: 10, and residues 20-145 of SEQ ID NO: 14. Nucleic acid sequences coding for the above VH domains are residues 58-426 of SEQ ID NO: 1, residues 58-426 of SEQ ID NO: 5, residues 58-426 of SEQ ID NO: 9 or residues 58-435 of SEQ ID NO: 13 The antibody or functional fragment thereof having the ability to bind to CA19-9 includes a VL domain that has an amino acid sequence selected from the group consisting of residues 20-130 of SEQ ID NO: 4, residues 20-129 of SEQ ID NO: 8, residues 20-130 of SEQ ID NO: 12, and residues 23-130 of SEQ ID NO: 16. Nucleic acid sequences coding for the above VL domains are residues 58-390 of SEQ ID NO: 3, residues 58-387 of SEQ ID NO: 7, residues 58-390 of SEQ ID NO: 11 or residues 67-390 of SEQ ID NO: 15.

In another embodiment, the antibody or functional fragment thereof that has the ability to bind to CA19-9 and to be useful in the methods of the present invention has one or more of the complementarity determining regions (CDRs) listed in Table 2. An antibody or functional fragment thereof that includes one or more of the CDRs can specifically bind to sLe^{a} as described herein.

TABLE 2
CDRs of Clonal Isolates

| | Nucleic Acid Residues | | | Amino Acid Residues | | |
|---|---|---|---|---|---|---|
| Variable | (SEQ ID NO:) | | | (SEQ ID NO:) | | |
| Domain | CDR1 | CDR2 | CDR3 | CDR1 | CDR2 | CDR3 |
| 5B1 VH | 133-156 | 208-231 | 346-393 | 55-62 | 70-77 | 116-131 |
| | (NO: 1) | (NO: 1) | (NO: 1) | (NO: 2) | (NO: 2) | (NO: 2) |
| 5B1 VL | 133-156 | 208-216 | 325-360 | 45-52 | 70-72 | 109-120 |
| | (NO: 3) | (NO: 3) | (NO: 3) | (NO: 4) | (NO: 4) | (NO: 4) |
| 9H3 VH | 133-156 | 208-231 | 346-393 | 45-52 | 70-77 | 116-131 |
| | (NO: 5) | (NO: 5) | (NO: 5) | (NO: 6) | (NO: 6) | (NO: 6) |
| 9H3 VL | 133-156 | 208-216 | 325-357 | 45-52 | 70-72 | 109-119 |
| | (NO: 7) | (NO: 7) | (NO: 7) | (NO: 8) | (NO: 8) | (NO: 8) |
| 5H11 VH | 133-156 | 208-231 | 346-393 | 45-52 | 70-77 | 116-131 |
| | (NO: 9) | (NO: 9) | (NO: 9) | (NO: 10) | (NO: 10) | (NO: 10) |
| 5H11 VL | 134-156 | 208-216 | 325-360 | 45-52 | 70-72 | 109-120 |
| | (NO: 11) | (NO: 11) | (NO: 11) | (NO: 12) | (NO: 12) | (NO: 12) |
| 7E3 VH | 133-156 | 208-231 | 346-402 | 45-52 | 70-77 | 116-134 |
| | (NO: 13) | (NO: 13) | (NO: 13) | (NO: 14) | (NO: 14) | (NO: 14) |
| 7E3 VK | 145-162 | 214-222 | 331-360 | 49-53 | 72-74 | 111-120 |
| | (NO: 15) | (NO: 15) | (NO: 15) | (NO: 16) | (NO: 16) | (NO: 16) |

In some embodiments, the antibody having the ability to bind to CA19-9 or functional fragment thereof includes less than six CDRs. In some embodiments, the antibody or functional fragment thereof includes one, two, three, four, or five CDRs selected from the group consisting of VH CDR1, VH CDR2, VH CDR3, VL CDR1, VL CDR2, and/or VL CDR3. In some embodiments, the antibody or functional fragment thereof includes one, two, three, four, or five CDRs selected from the group consisting of VH CDR1, VH CDR2, VH CDR3, VL CDR1, VL CDR2, and/or VL CDR3 of clonal isolates 5B1, 9H3, 5H11 or 7E3 described herein.

In some embodiments, the antibody having the ability to bind to CA 19-9 or functional fragment thereof includes a variable heavy (VH) chain domain having the CDR1, CDR2 and CDR3 amino acid sequence of the clonal isolate 5B1, 9H3, 5H11 or 7E3. Such VH domains can include the amino acid residues 55-62, 70-77 and 116-131 of SEQ ID NO: 2, or alternatively the amino acid residues 45-52, 70-77 and 116-131 of SEQ ID NO: 6, or alternatively the amino acid residues 45-52, 70-77 and 116-131 of SEQ ID NO: 10, or alternatively the amino acid residues 45-52, 70-77 and 116-134 of SEQ ID NO: 14. In another aspect, the nucleotide sequence encoding the CDR1, CDR2 and CDR3 of the VH domain can respectively include the nucleotide sequence of residues 133-156, 208-231 and 346-393 of SEQ ID NO: 1, or alternatively the nucleotide sequence of residues 133-156, 208-231 and 346-393 of SEQ ID NO: 5, or alternatively the nucleotide sequence of residues 133-156, 208-231 and 346-393 of SEQ ID NO: 9, or alternatively the nucleotide sequence of residues 133-156, 208-231, 346-402 of SEQ ID NO: 13.

In further preferred embodiments, an antibody having the ability of binding to CA19-9 preferably comprises at least the CDR3 variable region, of the heavy chain variable region (VH) and/or of the light chain variable region (VL) of a monoclonal antibody against CA19-9, preferably of a monoclonal antibody against CA19-9 described herein, and preferably comprises one or more of the complementarity-determining regions (CDRs), preferably at least the CDR3 variable region, of the heavy chain variable regions (VH) and/or light chain variable regions (VL) described herein.

In one embodiment, an antibody comprising one or more CDRs, a set of CDRs or a combination of sets of CDRs as described herein comprises said CDRs together with their intervening framework regions. Preferably, the portion will also include at least about 50% of either or both of the first and fourth framework regions, the 50% being the C-terminal 50% of the first framework region and the N-terminal 50% of the fourth framework region. Construction of antibodies made by recombinant DNA techniques may result in the introduction of residues N- or C-terminal to the variable regions encoded by linkers introduced to facilitate cloning or other manipulation steps, including the introduction of linkers to join variable regions of the invention to further protein sequences including immunoglobulin heavy chains, other variable domains (for example in the production of diabodies) or protein labels.

In one embodiment an antibody comprising one or more CDRs, a set of CDRs or a combination of sets of CDRs as described herein comprises said CDRs in a human antibody framework.

Reference herein to an antibody comprising with respect to the heavy chain thereof a particular chain, or a particular region or sequence preferably relates to the situation wherein all heavy chains of said antibody comprise said particular chain, region or sequence. This applies correspondingly to the light chain of an antibody.

The term “nucleic acid”, as used herein, is intended to include DNA and RNA. A nucleic acid may be single-stranded or double-stranded, but preferably is double-stranded DNA. The term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. Thus, the terms “nucleotide sequence” or “nucleic acid sequence” is the alphabetical representation of a polynucleotide. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules. Unless otherwise specified or required, any embodiment of this invention that is a polynucleotide encompasses both the double-stranded form and each of two complementary single-stranded forms known or predicted to make up the double-stranded form. It is understood that the isolated polynucleotides and nucleic acids described herein are directed to non-naturally occurring polynucleotides and nucleic acids. Non-naturally occurring polynucleotides and nucleic acids can include, but are not limited to, cDNA and chemically synthesized molecules.

The term “encode” or grammatical equivalents thereof as it is used in reference to polynucleotides refers to a polynucleotide in its native state or when manipulated by methods well known to those skilled in the art that can be transcribed to produce mRNA, which is then translated into a polypeptide and/or a fragment thereof. The antisense strand is the complement of such a polynucleotide, and the encoding sequence can be deduced therefrom.

According to the invention, the term “expression” is used in its most general meaning and comprises the production of RNA or of RNA and protein/peptide. It also comprises partial expression of nucleic acids. Furthermore, expression may be carried out transiently or stably.

The teaching given herein with respect to specific amino acid sequences, e.g. those shown in the sequence listing, is to be construed so as to also relate to variants of said specific sequences resulting in sequences which are functionally equivalent to said specific sequences, e.g. amino acid sequences exhibiting properties identical or similar to those of the specific amino acid sequences. One important property is to retain binding of an antibody to its target or to sustain effector functions of an antibody. Preferably, a sequence which is a variant with respect to a specific sequence, when it replaces the specific sequence in an antibody retains binding of said antibody to CA19-9 and preferably functions of said antibody as described herein, e.g. CDC mediated lysis or ADCC mediated lysis.

It will be appreciated by those skilled in the art that in particular the sequences of the CDR, hypervariable and variable regions can be modified without losing the ability to bind CA19-9. For example, CDR regions will be either identical or highly homologous to the regions of antibodies specified herein. By “highly homologous” it is contemplated that from 1 to 5, preferably from 1 to 4, such as 1 to 3 or 1 or 2 substitutions may be made in the CDRs. In addition, the hypervariable and variable regions may be modified so that they show substantial homology with the regions of antibodies specifically disclosed herein.

For the purposes of the present invention, “variants” of an amino acid sequence comprise amino acid insertion variants, amino acid addition variants, amino acid deletion variants and/or amino acid substitution variants. Amino acid deletion variants that comprise the deletion at the N-terminal and/or C-terminal end of the protein are also called N-terminal and/or C-terminal truncation variants.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible.

Amino acid addition variants comprise amino- and/or carboxy-terminal fusions of one or more amino acids, such as 1, 2, 3, 5, 10, 20, 30, 50, or more amino acids.

Amino acid deletion variants are characterized by the removal of one or more amino acids from the sequence, such as by removal of 1, 2, 3, 5, 10, 20, 30, 50, or more amino acids. The deletions may be in any position of the protein.

Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids.

Preferably, the degree of similarity, preferably identity between a given amino acid sequence and an amino acid sequence which is a variant of said given amino acid sequence will be at least about 60%, 65%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, preferably continuous amino acids. In preferred embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences.

The term “percentage identity” is intended to denote a percentage of amino acid residues which are identical between the two sequences to be compared, obtained after the best alignment, this percentage being purely statistical and the differences between the two sequences being distributed randomly and over their entire length. Sequence comparisons between two amino acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The percentage identity is calculated by determining the number of identical positions between the two sequences being compared, dividing this number by the number of positions compared and multiplying the result obtained by 100 so as to obtain the percentage identity between these two sequences.

The term “transgenic animal” refers to an animal having a genome comprising one or more transgenes, preferably heavy and/or light chain transgenes, or transchromosomes (either integrated or non-integrated into the animal's natural genomic DNA) and which is preferably capable of expressing the transgenes. For example, a transgenic mouse can have a human light chain transgene and either a human heavy chain transgene or human heavy chain transchromosome, such that the mouse produces human anti-CA19-9 antibodies when immunized with CA19-9 antigen and/or cells expressing CA19-9. The human heavy chain transgene can be integrated into the chromosomal DNA of the mouse, as is the case for transgenic mice, e.g., HuMAb mice, such as HCo7 or HCo12 mice, or the human heavy chain transgene can be maintained extrachromosomally, as is the case for transchromosomal (e.g., KM) mice as described in International Patent Application Publication No. WO 02/43478. Such transgenic and transchromosomal mice may be capable of producing multiple isotypes of human monoclonal antibodies to CA19-9 (e.g., IgG, IgA and/or IgE) by undergoing V-D-J recombination and isotype switching.

“Reduce”, “decrease” or “inhibit” as used herein means an overall decrease or the ability to cause an overall decrease, preferably of 5% or greater, 10% or greater, 20% or greater, more preferably of 50% or greater, and most preferably of 75% or greater, in the level, e.g. in the level of expression or in the level of proliferation of cells.

Terms such as “increase” or “enhance” preferably relate to an increase or enhancement by about at least 10%, preferably at least 20%, preferably at least 30%, more preferably at least 40%, more preferably at least 50%, even more preferably at least 80%, and most preferably at least 100%, at least 200%, at least 500%, at least 1000%, at least 10000% or even more.

Mechanisms of mAb Action

Although the following provides considerations regarding the mechanism underlying the therapeutic efficacy of antibodies of the invention it is not to be considered as limiting to the invention in any way.

The antibodies described herein preferably interact with components of the immune system, preferably through ADCC or CDC. Antibodies described herein can also be used to target payloads (e.g., radioisotopes, drugs or toxins) to directly kill tumor cells or can be used synergistically with traditional chemotherapeutic agents, attacking tumors through complementary mechanisms of action that may include anti-tumor immune responses that may have been compromised owing to a chemotherapeutic's cytotoxic side effects on T lymphocytes. However, antibodies described herein may also exert an effect simply by binding to CA19-9 on the cell surface, thus, e.g. blocking proliferation of the cells.

Antibody-Dependent Cell-Mediated Cytotoxicity

ADCC describes the cell-killing ability of effector cells as described herein, in particular lymphocytes, which preferably requires the target cell being marked by an antibody.

ADCC preferably occurs when antibodies bind to antigens on tumor cells and the antibody Fe domains engage Fc receptors (FcR) on the surface of immune effector cells. Several families of Fc receptors have been identified, and specific cell populations characteristically express defined Fe receptors. ADCC can be viewed as a mechanism to directly induce a variable degree of immediate tumor destruction that leads to antigen presentation and the induction of tumor-directed T-cell responses. Preferably, in vivo induction of ADCC will lead to tumor-directed T-cell responses and host-derived antibody responses.

Complement-Dependent Cytotoxicity

CDC is another cell-killing method that can be directed by antibodies. IgM is the most effective isotype for complement activation. IgG1 and IgG3 are also both very effective at directing CDC via the classical complement-activation pathway. Preferably, in this cascade, the formation of antigen-antibody complexes results in the uncloaking of multiple C1q binding sites in close proximity on the C_{H}2 domains of participating antibody molecules such as IgG molecules (C1q is one of three subcomponents of complement C1). Preferably these uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a. Preferably, the complement cascade ends in the formation of a membrane attack complex, which creates pores in the cell membrane that facilitate free passage of water and solutes into and out of the cell.

Production and Characterization of Antibodies

Antibodies described herein and useful in the methods described herein can be produced by a variety of techniques, including conventional monoclonal antibody methodology, e.g., the standard somatic cell hybridization technique of Kohler and Milstein, 1975, Nature 256:495. Although somatic cell hybridization procedures are preferred, in principle, other techniques for producing monoclonal antibodies can be employed, e.g., viral or oncogenic transformation of B-lymphocytes or phage display techniques using libraries of antibody genes.

In some embodiments, an animal system for preparing hybridomas that secrete monoclonal antibodies may be a murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Other preferred animal systems for preparing hybridomas that secrete monoclonal antibodies are the rat and the rabbit system (e.g. described in Spieker-Polet et al., 1995, Proc. Natl. Acad. Sci. U.S.A. 92:9348; see also Rossi et al., 2005, Am. J. Clin. Pathol. 124: 295).

In yet another preferred embodiment, human monoclonal antibodies can be generated using transgenic or transchromosomal mice carrying parts of the human immune system rather than the mouse system. These transgenic and transchromosomic mice include mice known as HuMAb mice and KM mice, respectively, and are collectively referred to herein as “transgenic mice.” The production of human antibodies in such transgenic mice can be performed as described in detail for CD20 in International Patent Application Publication No. WO 2004/035607.

Yet another strategy for generating monoclonal antibodies is to directly isolate genes encoding antibodies from lymphocytes producing antibodies of defined specificity. For details of recombinant antibody engineering see also Welschof and Kraus, Recombinant antibodes for cancer therapy ISBN-0-89603-918-8 and Benny K. C. Lo Antibody Engineering ISBN 1-58829-092-1.

To generate antibodies, mice can be immunized with carrier-conjugated peptides derived from the antigen sequence, i.e., the sequence against which the antibodies are to be directed, an enriched preparation of recombinantly expressed antigen or fragments thereof and/or cells expressing the antigen, as described. Alternatively, mice can be immunized with DNA encoding the antigen or fragments thereof. In the event that immunizations using a purified or enriched preparation of the antigen do not result in antibodies, mice can also be immunized with cells expressing the antigen, e.g., a cell line, to promote immune responses.

The immune response can be monitored over the course of the immunization protocol with plasma and serum samples being obtained by tail vein or retroorbital bleeds. Mice with sufficient titers of immunoglobulin can be used for fusions. Mice can be boosted intraperitonealy or intravenously with antigen expressing cells 3 days before sacrifice and removal of the spleen to increase the rate of specific antibody secreting hybridomas.

To generate hybridomas producing monoclonal antibodies, splenocytes and lymph node cells from immunized mice can be isolated and fused to an appropriate immortalized cell line, such as a mouse myeloma cell line. The resulting hybridomas can then be screened for the production of antigen-specific antibodies. Individual wells can then be screened by ELISA for antibody secreting hybridomas. By Immunofluorescence and FACS analysis using antigen expressing cells, antibodies with specificity for the antigen can be identified. The antibody secreting hybridomas can be re-plated, screened again, and if still positive for monoclonal antibodies can be subcloned by limiting dilution. The stable subclones can then be cultured in vitro to generate antibody in tissue culture medium for characterization.

Antibodies also can be produced in a host cell transfectoma using, for example, a combination of recombinant DNA techniques and gene transfection methods as are well known in the art (Morrison, 1985, Science 229:1202).

For example, in one embodiment, the gene(s) of interest, e.g., antibody genes, can be ligated into an expression vector such as a eukaryotic expression plasmid such as used by the GS gene expression system disclosed in International Patent Application Publication Nos. WO 87/04462 and WO 89/01036 and EP 338 841 A or other expression systems well known in the art. The purified plasmid with the cloned antibody genes can be introduced in eukaryotic host cells such as CHO cells, NS/0 cells, HEK293T cells or HEK293 cells or alternatively other eukaryotic cells like plant derived cells, fungal or yeast cells. The method used to introduce these genes can be methods described in the art such as electroporation, lipofectine, lipofectamine or others. After introduction of these antibody genes in the host cells, cells expressing the antibody can be identified and selected. These cells represent the transfectomas which can then be amplified for their expression level and upscaled to produce antibodies. Recombinant antibodies can be isolated and purified from these culture supernatants and/or cells.

Alternatively, the cloned antibody genes can be expressed in other expression systems, including prokaryotic cells, such as microorganisms, e.g. E. coli. Furthermore, the antibodies can be produced in transgenic non-human animals, such as in milk from sheep and rabbits or in eggs from hens, or in transgenic plants; see e.g. Verma, R., et al. (1998) J. Immunol. Meth. 216: 165-181; Pollock, et al. (1999) J. Immunol. Meth. 231: 147-157; and Fischer, R., et al. (1999) Biol. Chem. 380: 825-839.

Chimerization

Murine monoclonal antibodies can be used as therapeutic antibodies in humans, for example when labeled with toxins or radioactive isotopes. Non-labeled murine antibodies can be highly immunogenic in man when repetitively applied leading to reduction of the therapeutic effect. The main immunogenicity is mediated by the heavy chain constant regions. The immunogenicity of murine antibodies in man can be reduced or completely avoided if respective antibodies are chimerized or humanized. Chimeric antibodies are antibodies, the different portions of which are derived from different animal species, such as those having a variable region derived from a murine antibody and a human immunoglobulin constant region. Chimerisation of antibodies is achieved by joining of the variable regions of the murine antibody heavy and light chain with the constant region of human heavy and light chain (e.g. as described by Kraus et al., in Methods in Molecular Biology series, Recombinant antibodies for cancer therapy ISBN-0-89603-918-8). In an embodiment, chimeric antibodies are generated by joining human kappa-light chain constant region to murine light chain variable region. In an also preferred embodiment chimeric antibodies can be generated by joining human lambda-light chain constant region to murine light chain variable region. The preferred heavy chain constant regions for generation of chimeric antibodies are IgG1, IgG3 and IgG4. Other preferred heavy chain constant regions for generation of chimeric antibodies are IgG2, IgA, IgD and IgM.

Humanization

Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., 1998, Nature 332:323-327; Jones et al., 1986, Nature 321:522-525; and Queen et al., 1989, Proc. Natl. Acad. Sci. U.S.A. 86:10029-10033). Such framework sequences can be obtained from public DNA databases that include germline antibody gene sequences. These germline sequences will differ from mature antibody gene sequences because they will not include completely assembled variable genes, which are formed by V (D) J joining during B cell maturation. Germline gene sequences will also differ from the sequences of a high affinity secondary repertoire antibody at individual evenly across the variable region.

The ability of antibodies to bind an antigen can be determined using standard binding assays (e.g., ELISA, Western Blot, Immunofluorescence and flow cytometric analysis).

To purify antibodies, selected hybridomas can be grown in two-liter spinner-flasks for monoclonal antibody purification. Alternatively, antibodies can be produced in dialysis-based bioreactors. Supernatants can be filtered and, if necessary, concentrated before affinity chromatography with protein G-sepharose or protein A-sepharose. Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C.

To determine if selected monoclonal antibodies bind to unique epitopes and/or to characterize one or more binding properties, site-directed or multi-site directed mutagenesis can be used.

To determine the isotype of antibodies, isotype ELISAs with various commercial kits (e.g. Zymed, Roche Diagnostics) can be performed. Wells of microtiter plates can be coated with anti-mouse Ig. After blocking, the plates are reacted with monoclonal antibodies or purified isotype controls, at ambient temperature for two hours. The wells can then be reacted with either mouse IgG1, IgG2a, IgG2b or IgG3, IgA or mouse IgM-specific peroxidase-conjugated probes. After washing, the plates can be developed with ABTS substrate (1 mg/ml) and analyzed at OD of 405-650. Alternatively, the IsoStrip Mouse Monoclonal Antibody Isotyping Kit (Roche, Cat. No. 1493027) may be used as described by the manufacturer.

In order to demonstrate presence of antibodies in sera of immunized mice or binding of monoclonal antibodies to living cells expressing antigen, flow cytometry can be used. Cell lines expressing naturally or after transfection antigen and negative controls lacking antigen expression (grown under standard growth conditions) can be mixed with various concentrations of monoclonal antibodies in hybridoma supernatants or in PBS containing 1% FBS, and can be incubated at 4° C. for 30 min. After washing, the APC- or Alexa647-labeled anti IgG antibody can bind to antigen-bound monoclonal antibody under the same conditions as the primary antibody staining. The samples can be analyzed by flow cytometry with a FACS instrument using light and side scatter properties to gate on single, living cells. In order to distinguish antigen-specific monoclonal antibodies from non-specific binders in a single measurement, the method of co-transfection can be employed. Cells transiently transfected with plasmids encoding antigen and a fluorescent marker can be stained as described above. Transfected cells can be detected in a different fluorescence channel than antibody-stained cells. As the majority of transfected cells express both transgenes, antigen-specific monoclonal antibodies bind preferentially to fluorescence marker expressing cells, whereas non-specific antibodies bind in a comparable ratio to non-transfected cells. An alternative assay using fluorescence microscopy may be used in addition to or instead of the flow cytometry assay. Cells can be stained exactly as described above and examined by fluorescence microscopy.

In order to demonstrate presence of antibodies in sera of immunized mice or binding of monoclonal antibodies to living cells expressing antigen, immunofluorescence microscopy analysis can be used. For example, cell lines expressing either spontaneously or after transfection antigen and negative controls lacking antigen expression are grown in chamber slides under standard growth conditions in DMEM/F12 medium, supplemented with 10% fetal calf serum (FCS), 2 mM L-glutamine, 100 IU/ml penicillin and 100 μg/ml streptomycin. Cells can then be fixed with methanol or paraformaldehyde or left untreated. Cells can then be reacted with monoclonal antibodies against the antigen for 30 min. at 25° C. After washing, cells can be reacted with an Alexa555-labelled anti-mouse IgG secondary antibody (Molecular Probes) under the same conditions. Cells can then be examined by fluorescence microscopy.

Cell extracts from cells expressing antigen and appropriate negative controls can be prepared and subjected to sodium dodecyl sulfate (SDS) polyacrylamide gel electrophoresis. After electrophoresis, the separated antigens will be transferred to nitrocellulose membranes, blocked, and probed with the monoclonal antibodies to be tested. IgG binding can be detected using anti-mouse IgG peroxidase and developed with ECL substrate.

Antibodies can be further tested for reactivity with antigen by Immunohistochemistry in a manner well known to the skilled person, e.g. using paraformaldehyde or acetone fixed cryosections or paraffin embedded tissue sections fixed with paraformaldehyde from non-cancer tissue or cancer tissue samples obtained from patients during routine surgical procedures or from mice carrying xenografted tumors inoculated with cell lines expressing spontaneously or after transfection antigen. For immunostaining, antibodies reactive to antigen can be incubated followed by horseradish-peroxidase conjugated goat anti-mouse or goat anti-rabbit antibodies (DAKO) according to the vendors instructions.

Antibodies can be tested for their ability to mediate phagocytosis and killing of cells expressing CA19-9. The testing of monoclonal antibody activity in vitro will provide an initial screening prior to testing in vivo models.

Antibody Dependent Cell-Mediated Cytotoxicity (ADCC)

Briefly, polymorphonuclear cells (PMNs), NK cells, monocytes, mononuclear cells or other effector cells, from healthy donors can be purified by Ficoll Hypaque density centrifugation, followed by lysis of contaminating erythrocytes. Washed effector cells can be suspended in RPMI supplemented with 10% heat-inactivated fetal calf serum or, alternatively with 5% heat-inactivated human serum and mixed with ⁵¹Cr labeled target cells expressing CA19-9, at various ratios of effector cells to target cells. Alternatively, the target cells may be labeled with a fluorescence enhancing ligand (BATDA). A highly fluorescent chelate of Europium with the enhancing ligand which is released from dead cells can be measured by a fluorometer. Another alternative technique may utilize the transfection of target cells with luciferase. Added lucifer yellow may then be oxidated by viable cells only. Purified anti-CA19-9 IgGs can then be added at various concentrations. Irrelevant human IgG can be used as negative control. Assays can be carried out for 4 to 20 hours at 37° C. depending on the effector cell type used. Samples can be assayed for cytolysis by measuring ⁵¹Cr release or the presence of the EuTDA chelate in the culture supernatant. Alternatively, luminescence resulting from the oxidation of lucifer yellow can be a measure of viable cells.

Anti-CA19-9 monoclonal antibodies can also be tested in various combinations to determine whether cytolysis is enhanced with multiple monoclonal antibodies.

Complement Dependent Cytotoxicity (CDC)

Monoclonal anti-CA19-9 antibodies can be tested for their ability to mediate CDC using a variety of known techniques. For example, serum for complement can be obtained from blood in a manner known to the skilled person. To determine the CDC activity of mAbs, different methods can be used. ⁵¹Cr release can for example be measured or elevated membrane permeability can be assessed using a propidium iodide (PI) exclusion assay. Briefly, target cells can be washed and 5×10⁵/ml can be incubated with various concentrations of mAb for 10-30 min. at room temperature or at 37° C. Serum or plasma can then be added to a final concentration of 20% (v/v) and the cells incubated at 37° C. for 20-30 min. All cells from each sample can be added to the PI solution in a FACS tube. The mixture can then be analyzed immediately by flow cytometry analysis using FACSArray.

In an alternative assay, induction of CDC can be determined on adherent cells. In one embodiment of this assay, cells are seeded 24 h before the assay with a density of 3×10⁴/well in tissue-culture flat-bottom microtiter plates. The next day growth medium is removed and the cells are incubated in triplicates with antibodies. Control cells are incubated with growth medium or growth medium containing 0.2% saponin for the determination of background lysis and maximal lysis, respectively. After incubation for 20 min. at room temperature supernatant is removed and 20% (v/v) human plasma or serum in DMEM (prewarmed to 37° C.) is added to the cells and incubated for another 20 min. at 37° C. All cells from each sample are added to propidium iodide solution (10 μg/ml). Then, supernatants are replaced by PBS containing 2.5 μg/ml ethidium bromide and fluorescence emission upon excitation at 520 nm is measured at 600 nm using a Tecan Safire. The percentage specific lysis is calculated as follows: % specific lysis=(fluorescence sample−fluorescence background)/(fluorescence maximal lysis−fluorescence background)×100.

Induction of Apoptosis and Inhibition of Cell Proliferation by Monoclonal Antibodies

To test for and/or assess the ability to initiate apoptosis, monoclonal anti-CA19-9 antibodies can, for example, be incubated with CA19-9-positive tumor cells at 37° C. for about 20 hours. The cells can be harvested, washed in Annexin-V binding buffer (BD biosciences), and incubated with Annexin V conjugated with FITC or APC (BD biosciences) for 15 min. in the dark. All cells from each sample can be added to PI solution (10 μg/ml in PBS) in a FACS tube and assessed immediately by flow cytometry (as above). Alternatively, a general inhibition of cell-proliferation by monoclonal antibodies can be detected with commercially available kits. The DELFIA Cell Proliferation Kit (Perkin-Elmer, Cat. No. AD0200) is a non-isotopic immunoassay based on the measurement of 5-bromo-2′-deoxyuridine (BrdU) incorporation during DNA synthesis of proliferating cells in microplates. Incorporated BrdU is detected using europium labelled monoclonal antibody. To allow antibody detection, cells are fixed and DNA denatured using Fix solution. Unbound antibody is washed away and DELFIA inducer is added to dissociate europium ions from the labelled antibody into solution, where they form highly fluorescent chelates with components of the DELFIA Inducer. The fluorescence measured—utilizing time-resolved fluorometry in the detection—is proportional to the DNA synthesis in the cell of each well.

Pharmaceutical Compositions

Compounds and agents described herein may be administered in the form of any suitable pharmaceutical composition.

Pharmaceutical compositions are usually provided in a uniform dosage form and may be prepared in a manner known per se. A pharmaceutical composition may e.g. be in the form of a solution or suspension.

A pharmaceutical composition may comprise salts, buffer substances, preservatives, carriers, diluents and/or excipients all of which are preferably pharmaceutically acceptable. The term “pharmaceutically acceptable” refers to the non-toxicity of a material which does not interact with the action of the active component of the pharmaceutical composition.

Salts which are not pharmaceutically acceptable may be used for preparing pharmaceutically acceptable salts and are included in the invention. Pharmaceutically acceptable salts of this kind comprise in a non-limiting way those prepared from the following acids: hydrochloric, hydrobromic, sulfuric, nitric, phosphoric, maleic, acetic, salicylic, citric, formic, malonic, succinic acids, and the like. Pharmaceutically acceptable salts may also be prepared as alkali metal salts or alkaline earth metal salts, such as sodium salts, potassium salts or calcium salts.

Suitable buffer substances for use in a pharmaceutical composition include acetic acid in a salt, citric acid in a salt, boric acid in a salt and phosphoric acid in a salt.

Suitable preservatives for use in a pharmaceutical composition include benzalkonium chloride, chlorobutanol, paraben and thimerosal.

An injectable formulation may comprise a pharmaceutically acceptable excipient such as Ringer Lactate.

The term “carrier” refers to an organic or inorganic component, of a natural or synthetic nature, in which the active component is combined in order to facilitate, enhance or enable application. According to the invention, the term “carrier” also includes one or more compatible solid or liquid fillers, diluents or encapsulating substances, which are suitable for administration to a patient.

Possible carrier substances for parenteral administration are e.g. sterile water, Ringer, Ringer lactate, sterile sodium chloride solution, polyalkylene glycols, hydrogenated naphthalenes and, in particular, biocompatible lactide polymers, lactide/glycolide copolymers or polyoxyethylene/polyoxy-propylene copolymers.

The term “excipient” when used herein is intended to indicate all substances which may be present in a pharmaceutical composition and which are not active ingredients such as, e.g., carriers, binders, lubricants, thickeners, surface active agents, preservatives, emulsifiers, buffers, flavoring agents, or colorants.

The agents and compositions described herein may be administered via any conventional route, such as by parenteral administration including by injection or infusion. Administration is preferably parenterally, e.g. intravenously, intraarterially, subcutaneously, intradermally or intramuscularly.

Compositions suitable for parenteral administration usually comprise a sterile aqueous or nonaqueous preparation of the active compound, which is preferably isotonic to the blood of the recipient. Examples of compatible carriers and solvents are Ringer solution and isotonic sodium chloride solution. In addition, usually sterile, fixed oils are used as solution or suspension medium.

In one embodiment, one or more antibody or functional fragment of the invention is in a liquid pharmaceutical formulation. Liquid pharmaceutically administrable compositions can, for example, be prepared by dissolving, dispersing, or otherwise mixing an antibody or functional fragment as provided herein and optional pharmaceutical adjuvants in a carrier, such as, for example, water, saline, aqueous dextrose, glycerol, glycols, ethanol, and the like, to thereby form a solution. If desired, the pharmaceutical composition to be administered can also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences (1990) Mack Publishing Co., Easton, PA.

The agents and compositions described herein are administered in effective amounts. An “effective amount” refers to the amount which achieves a desired reaction or a desired effect alone or together with further doses. An effective dose may be an amount that achieves the desired reaction or desired effect, e.g., intended therapeutic result, when administered in accordance with a treatment regimen. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition.

An effective amount of an agent or composition described herein may depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the agents described herein may depend on various of such parameters. In the case that a reaction in a patient is insufficient with an initial dose, higher doses (or effectively higher doses achieved by a different, more localized route of administration) may be used.

In one embodiment, a therapeutically effective dosage produces a serum concentration of an antibody or functional fragment of from about 0.1 ng/ml to about 50-100 μg/ml. The pharmaceutical compositions, in another embodiment, provide a dosage of from about 0.001 mg to about 500 mg of antibody per kilogram of body weight per day. Pharmaceutical dosage unit forms can be prepared to provide from about 0.01 mg, 0.1 mg or 1 mg to about 30 mg, 100 mg or 500 mg, and in one embodiment from about 10 mg to about 500 mg of the antibody or functional fragment and/or a combination of other optional essential ingredients per dosage unit form.

The antibody or functional fragment of the invention can be administered at once, or may be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens can be adjusted over time according to the individual need and the professional judgment of the person administering or supervising the administration of the compositions, and that the concentration ranges set forth herein are exemplary only and are not intended to limit the scope or practice of the claimed compositions.

The agents and compositions described herein can be administered to patients, e.g., in vivo, to treat or prevent a variety of disorders such as those described herein. Preferred patients include human patients having disorders that can be corrected or ameliorated by administering the agents and compositions described herein. This includes disorders involving cells characterized by an altered expression pattern of CA19-9.

For example, in one embodiment, antibodies described herein can be used to treat a patient with a cancer disease, e.g., a cancer disease such as described herein characterized by the presence of cancer cells expressing CA19-9.

It is understood that modifications which do not substantially affect the activity of the various embodiments of this invention are also provided within the definition of the invention provided herein. Accordingly, the following examples are intended to illustrate but not limit the present invention.

### FIGURE

FIG. 1 depicts the administration schedule of the combination clinical trial, in which pancreatic cancer patients are administered both an antibody that binds to CA19-9 and FOLFIRINOX.

### EXAMPLE

Regimen for Treating Pancreatic Cancer with FOLFIRINOX and an Antibody Having the Ability to Bind to CA19-9.

A clinical study is performed comparing the therapeutic effect of the use of the MVT-5873 monoclonal antibody (5B1 monoclonal antibody) as a monotherapy or in combination with FOLFIRINOX in the treatment of pancreatic cancer, in patients with locally-advanced or metastatic pancreatic ductal adenocarcinoma (PDAC). Patients having other CA19-9-positive malignancies may also be included in the study.

The patients will be subdivided into the following groups:
Group A: A Q4 week monotherapy schedule employing a single dose of MVT 5873 administered 7 days prior to Cycle 1 Day 1
Group B: A Q2 week monotherapy schedule employing a single dose of MVT 5873 administered 7 days prior to Cycle 1 Day 1
Group C: A Q2 week schedule in combination with mFOLFIRINOX
   mFOLFIRINOX is a dose amount of FOLFIRINOX, in which FOLFIRINOX is administered at a dose of 65 mg/m² oxaliplatin, 400 mg/m² leucovorin, 150 mg/m² irinotecan, and 1200 mg/m² 5-fluorouracil.

In certain groups, the patients, after completing the combination therapy will be administered the antibody having the ability to bind CA 19-9 as a monotherapy.

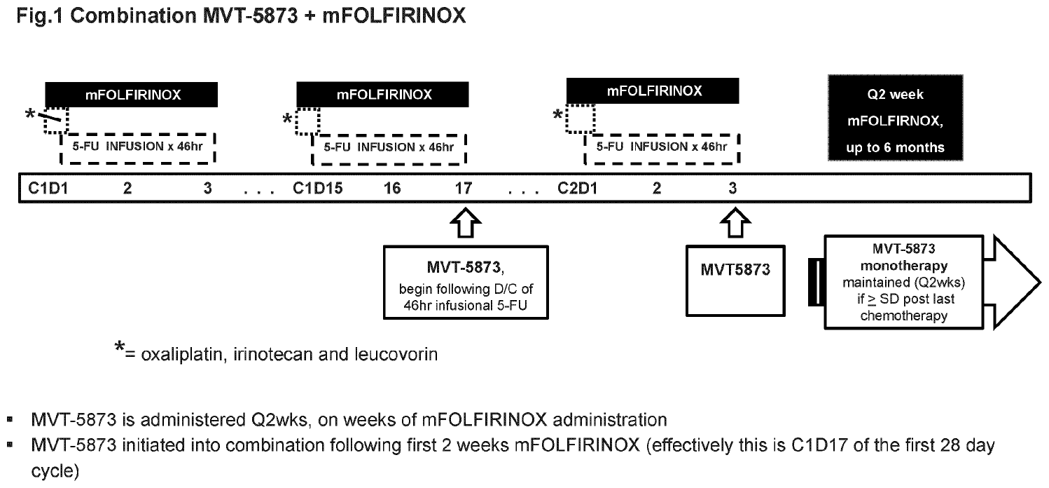

The administration schedule for Group C is depicted in FIG. 1.

A number of patients in Group C have been administered MVT 5873 at 0.5 mg/kg in combination with mFOLFIRINOX (65 mg/m² oxaliplatin, 400 mg/m² leucovorin, 150 mg/m² irinotecan, and 1200 mg/m² 5-fluorouracil). One patient received six cycles of therapy as depicted in FIG. 1 and treatment is ongoing (under treatment for more than 180 days). Another patient received two cycles of therapy as depicted in FIG. 1 (under treatment for more than 50 days), and another patient received a single cycle of therapy as depicted in FIG. 1 (under treatment for more than 20 days). No dose limiting toxicities have been reported for these patients.

The results from this trial will show that the combination treatment with an antibody that binds to CA19-9 and FOLFIRINOX surprisingly provides a more than additive, synergistic, therapeutic effect compared to either the antibody or FOLFIRINOX treatment as a monotherapy.

We claim:

1. A method of treating or preventing a CA19-9-positive cancer in a patient comprising administering to the patient an antibody having the ability to bind to CA19-9 in combination with the administration of FOLFIRINOX.

2. The method of claim 1, wherein the antibody having the ability to bind to CA19-9 is administered repeatedly at a dose of up to 100 mg/kg.

3. The method of claim 1 or 2, wherein the antibody having the ability to bind to CA19-9 is administered repeatedly at a dose of 0.01 to 10 mg/kg.

4. The method of any one of claims 1 to 3, wherein the antibody having the ability to bind to CA19-9 is administered repeatedly at a dose of 0.5 to 1.0 mg/kg.

5. The method of any one of claims 1 to 4, wherein the antibody having the ability to bind to CA19-9 is administered once a week, once every two weeks, once every six weeks, or once every two months.

6. The method of any one of claims 1 to 5, wherein the antibody having the ability to bind to CA19-9 is administered once every two weeks.

7. The method of any one of claims 1 to 6, wherein FOLFIRINOX comprises oxaliplatin, leucovorin, irinotecan and 5-fluorouracil.

8. The method of any one of claims 1 to 7, wherein FOLFIRINOX is administered at a dose of 65 mg/m² oxaliplatin, 400 mg/m² leucovorin, 150 mg/m² irinotecan, and 1200 mg/m² 5-fluorouracil.

9. The method of any one of claims 1 to 8, wherein the antibody having the ability to bind to CA19-9 is administered in an amount of 0.5 to 1.0 mg/kg once every two weeks starting on day 1 and FOLFIRINOX is administered intravenously at a dose of 65 mg/m² oxaliplatin on day 1, 400 mg/m² leucovorin on day 1, 150 mg/m² irinotecan on day 1, and a total of 1200 mg/m² 5-fluorouracil over days 1 and 2.

10. The method of any one of claims 1 to 9, wherein the method further comprises administering to the patient concurrently or successively one or more additional agents.

11. The method of claim 10, wherein the additional agent is a chemotherapeutic agent selected from the group consisting of gemcitabine, paclitaxel, prodrugs thereof, salts thereof, and combinations thereof.

12. The method of claim 10, wherein the additional agent is an immunotherapeutic agent, preferably an agent capable of stimulating γδ T cells, wherein the γδ T cells are preferably Vγ9Vδ2 T cells.

13. The method of claim 12, wherein the agent capable of stimulating γδ T cells is a bisphosphonate.

14. The method of claim 12 or 13, wherein the agent capable of stimulating γδ T cells is a nitrogen-containing bisphosphonate (aminobisphosphonate).

15. The method of any one of claims 12 to 14, wherein the agent capable of stimulating γδ T cells is selected from the group consisting of zoledronic acid, clodronic acid, ibandronic acid, pamidronic acid, risedronic acid, minodronic acid, olpadronic acid, alendronic acid, incadronic acid and salts thereof.

16. The method of any one of claims 12 to 15, wherein the agent capable of stimulating γδ T cells is administered in combination with interleukin-2.

17. The method of any one of claims 1 to 16, wherein the antibody having the ability to bind to CA19-9 mediates cell killing by one or more of complement dependent cytotoxicity (CDC) mediated lysis, antibody dependent cellular cytotoxicity (ADCC) mediated lysis, induction of apoptosis and inhibition of proliferation.

18. The method of any one of claims 1 to 17, wherein the antibody having the ability to bind to CA19-9 is a human antibody.

19. The method of any one of claims 1 to 18, wherein the antibody having the ability to bind to CA19-9 is an antibody binding fragment selected from the group consisting of a Fab, a Fab′, a F(ab′)₂, a scFV, a diabody, a triabody, a minibody and a single-domain antibody (sdAB).

20. The method of any one of claims 1 to 19, wherein the antibody having the ability to bind to CA19-9 is a diabody, preferably comprising the amino acid sequence of SEQ ID NO: 18 or 20.

21. The method of any one of claims 1 to 20, wherein the antibody having the ability to bind to CA19-9 is a monoclonal antibody or a chimeric antibody.

22. The method of any one of claims 1 to 21, wherein the antibody having the ability to bind to CA19-9 is an IgG or IgM isotype.

23. The method of any one of claims 1 to 22, wherein the antibody having the ability to bind to CA19-9 is an IgG 1 subclass.

24. The method of any one of claims 1 to 23, wherein the antibody having the ability to bind to CA19-9 is an antibody conjugate, wherein the antibody conjugate comprises an antibody or fragment thereof having the ability to bind to CA19-9 covalently conjugated or recombinantly fused to another moiety.

25. The method of claim 24, wherein the moiety is a stabilizing agent, a diagnostic agent, a detectable agent or a therapeutic agent.

26. The method of any one of claims 1 to 25, wherein the antibody having the ability to bind to CA19-9 comprises a variable heavy chain (VH) domain having an amino acid sequence selected from the group consisting of residues 20-142 of SEQ ID NO: 2, residues 20-142 of SEQ ID NO: 6, residues 20-142 of SEQ ID NO: 10, and residues 20-145 of SEQ ID NO: 14.

27. The method of any one of claims 1 to 26, wherein the antibody having the ability to bind to CA19-9 comprises a variable light chain (VL) domain having an amino acid sequence selected from the group consisting of residues 20-130 of SEQ ID NO: 4, residues 20-129 of SEQ ID NO: 8, residues 20-130 of SEQ IP NO: 12, and residues 23-130 of SEQ ID NO: 16.

28. The method of any one of claims 1 to 27, wherein the antibody having the ability to bind to CA19-9 comprises a variable heavy chain (VH) domain and a variable light chain (VL) domain, where said VH domain and said VL domain respectively comprise an amino acid sequence selected from the group consisting of residues 20-142 of SEQ ID NO: 2 and residues 20-130 of SEQ ID NO: 4; residues 20-142 of SEQ ID NO: 6 and residues 20-129 of SEQ ID NO: 8; residues 20-142 of SEQ ID NO: 10 and residues 20-130 of SEQ ID NO: 12; and residues 20-145 of SEQ ID NO: 14 and residues 23-130 of SEQ ID NO: 16.

29. The method of any one of claims 1 to 28, wherein the antibody having the ability to bind to CA19-9 comprises a variable heavy chain (VH) domain comprising the amino acid sequence of residues 20-142 of SEQ ID NO: 2 and a variable light chain (VL) domain comprising the amino acid sequence of residues 20-130 of SEQ ID NO: 4.

30. The method of any one of claims 1 to 29, wherein the antibody having the ability to bind to CA19-9 is an antibody selected from the group consisting of (i) an antibody which is a chimerized or humanized form of the antibody defined in any one of claims 26 to 29, (ii) an antibody having the specificity of the antibody defined in any one of claims 26 to 29, and (iii) an antibody comprising the antigen binding portion or antigen binding site, in particular the variable region, of the antibody defined in any one of claims 26 to 29 and preferably having the specificity of the antibody defined in any one of claims 26 to 29.

31. The method of any one of claims 1 to 30, wherein the antibody having the ability to bind to CA19-9 binds to the Sialyl Lewis A (sLe^{a}) antigen epitope present on CA19-9.

32. The method of any one of claims 1 to 31, wherein expression of CA19-9 is on the cell surface of a cancer cell.

33. The method of any one of claims 1 to 32, wherein the CA19-9-positive cancer is pancreatic cancer.

34. The method of claim 33, wherein the pancreatic cancer comprises primary cancer, advanced cancer or metastatic cancer, or a combination thereof such as a combination of pancreatic primary cancer and metastatic cancer.

35. The method of claim 34, wherein the metastatic cancer comprises metastasis to the lymph nodes, ovary, liver or lung, or a combination thereof.

36. The method of any one of claims 33 to 35, wherein the pancreatic cancer comprises a cancer of the pancreatic duct.

37. The method of any one of claims 33 to 36, wherein the pancreatic cancer comprises an adenocarcinoma or carcinoma, or a combination thereof.

38. The method of any one of claims 33 to 37, wherein the pancreatic cancer comprises a ductal adenocarcinoma, a mucinous adenocarcinoma, a neuroendocrine carcinoma or an acinic cell carcinoma, or a combination thereof.

39. The method of any one of claims 33 to 38, wherein the pancreatic cancer is partially or completely refractory to gemcitabine treatment such as gemcitabine monotherapy.

40. The method of any one of claims 33 to 39, wherein the pancreatic cancer is advanced or metastatic pancreatic ductal carcinoma (PDAC).

41. The method of any one of claims 33 to 40, wherein preventing pancreatic cancer comprises preventing a recurrence of pancreatic cancer.

42. The method of any one of claims 1 to 41, wherein the patient has a precancerous pancreatic lesion, in particular a precancerous pancreatic lesion comprising a beginning malignant histological change in the pancreatic ducts.

43. The method of any one of claims 1 to 42, wherein the patient has had surgery for the CA19-9-positive cancer.

44. The method of any one of claims 1 to 43, wherein the patient has circulating levels of CA19-9 of less than 4000 U/mL, preferably less than 1000 U/ml.

45. The method of any one of claims 1 to 44, wherein the patient has circulating levels of CA19-9 of 37 U/ml or less, or no detectable circulating levels of CA19-9.

46. A medical preparation for treating or preventing a CA19-9-positive cancer comprising (i) an antibody having the ability to bind to CA19-9 and (ii) FOLFIRINOX.

47. The medical preparation of claim 46, which is present in the form of a kit comprising a first container including the antibody having the ability to bind to CA19-9 and a second container including FOLFIRINOX or one or more of the agents comprising FOLFIRINOX.

48. The medical preparation of claim 46 or 47, further including printed instructions for use of the preparation for treatment or prevention of the CA19-9-positive cancer.
